(19) United States
(12) Patent Application Publication
(10) Pub. No.: US 2023/0173045 A1
(43) Pub. Date: Jun. 8, 2023

(54) RANKING NEOANTIGENS FOR PERSONALIZED CANCER VACCINE

(71) Applicant: Amazon Technologies, Inc., Reno, NV (US)

(72) Inventors: Layne Christopher PRICE, Seattle, WA (US); David HECKERMAN, Bellevue, WA (US); Frank Wilhelm SCHMITZ, Seattle, WA (US); Jasleen Kaur Grewal, Vancouver (CA); Anta IMATA SAFO, Seattle, WA (US)

(21) Appl. No.: 17/764,074

(22) PCT Filed: Feb. 4, 2022
(86) PCT No.: PCT/US2022/015275

§ 371 Date: Mar. 25, 2022

Related U.S. Application Data
(60) Provisional application No. 63/146,392, filed on Feb. 5, 2021.

(51) Int. Cl. A61K 39/00; G16B 20/20; G16B 40/20

(52) U.S. Cl. CPC A61K 39/0011; G16B 20/20; G16B 40/20; A61K 2039/575

(57) ABSTRACT

Disclosed herein are methods for ranking tumor-specific neoantigens from a tumor of a subject that are suitable for subject-specific immunogenic compositions. Suitable tumor-specific neoantigens are tumor-specific neoantigens that are likely presented on the cell surface of the tumor, are likely to be immunogenic, are predicted to be expressed in sufficient amounts to elicit an immune response in the subject, optionally represent sufficient diversity across the tumor, and have relatively high manufacture feasibility. The present methods take a set of neoantigens (peptide vaccine candidates) and ranks the neoantigens in a way such that a group of top-ranked neoantigens simultaneously promotes cell-surface presentation of important neoantigens for Class I and Class II MHC molecules. The top-ranked neoantigens can then be further narrowed according manufacturability and/or other criteria.

The present application claims the benefit of U.S. Provisional Application No. 63/146,392 filed on Feb. 5, 2021, the entire contents of which are incorporated herein by reference.

### REFERENCE TO A SEQUENCE LISTING

This application contains a Sequence Listing in computer readable form. The computer readable form is incorporated herein by reference. Said ASCII copy, created on Feb. 3, 2022, is named 146401_091686_SL.txt and is 14,005 bytes in size.

### BACKGROUND

Cancer is a leading cause of death worldwide accounting for 1 in 4 of all deaths. Siegel et al., CA: A Cancer Journal for Clinicians, 68:7-30 (2018). There were 18.1 million new cancer cases and 9.6 million cancer-related deaths in 2018. Bray et al., CA: A Cancer Journal for Clinicians, 68(6):394-424. There are a number of existing standard of care cancer therapies, including ablation techniques (e.g., surgical procedures and radiation) and chemical techniques (e.g., chemotherapeutic agents). Unfortunately, such therapies are frequently associated with serious risk, toxic side effects, and extremely high costs, as well as uncertain efficacy.

Cancer immunotherapy (e.g., cancer vaccine) has emerged as a promising cancer treatment modality. The goal of cancer immunotherapy is to harness the immune system for selective destruction of cancer while leaving normal tissues unharmed. Traditional cancer vaccines typically target tumor-associated antigens. Tumor-associated antigens are typically present in normal tissues, but overexpressed in cancer. However, because these antigens are often present in normal tissues immune tolerance can prevent immune activation. Several clinical trials targeting tumor-associated antigens have failed to demonstrate a durable beneficial effect compared to standard of care treatment. Li et al., Ann Oncol., 28 (Suppl 12): xii11-xii17 (2017).

Neoantigens represent an attractive target for cancer immunotherapies. Neoantigens are non-autologous proteins with individual specificity. Neoantigens are derived from random somatic mutations in the tumor cell genome and are not expressed on the surface of normal cells. Id. Because neoantigens are expressed exclusively on tumor cells, and thus do not induce central immune tolerance, cancer vaccines targeting cancer neoantigens have potential advantages, including decreased central immune tolerance and improved safety profile. Id.

The mutational landscape of cancer is complex and tumor mutations are generally unique to each individual subject. Most somatic mutations detected by sequencing do not result in effective neoantigens. Only a small percentage of mutations in the tumor DNA, or a tumor cell, are transcribed, translated, and processed into a tumor-specific neoantigen with sufficient accuracy to design a vaccine that is likely to be effective. Further, not all neoantigens are immunogenic. In fact, the proportion of T cells spontaneously recognizing endogenous neoantigens is about 1% to 2%. See, Karpanen et al., Front Immunol., 8:1718 (2017). Moreover, the cost and time associated with the manufacture of neoantigen vaccines is significant.

Thus, it remains a challenge to efficiently and accurately predict, prioritize, and select neoantigen candidates for immunogenic compositions. Accordingly, there is a significant unmet need for an integrated method to characterize tumor genomic material to identify neoantigens, identify which neoantigens are targeted by the immune system, and select which neoantigens are likely to be suitable for effective immunogenic compositions.

### SUMMARY

This disclosure relates to a novel method for ranking one or more suitable tumor-specific neoantigens from a tumor of a subject for a personalized (i.e. subject-specific) immunogenic composition. The disclosure also relates to methods of treating cancer in a subject in need thereof by administering an immunogenic composition comprising tumor-specific neoantigens selected using the novel approach for ranking tumor-specific neoantigens and formulating an immunogenic composition comprising tumor-specific neoantigens selected based on the present ranking technique. Suitable tumor-specific neoantigens are neoantigens that are likely presented on the cell surface of a tumor, are likely to be immunogenic, are predicted to be expressed in sufficient amounts to elicit an immune response in the subject, optionally represent sufficient diversity across the tumor, and have relatively high manufacture feasibility. The present methods take a set of neoantigens (peptide vaccine candidates) and rank the neoantigens in a way such that a group of top-ranked neoantigens simultaneously promotes cell-surface presentation of important neoantigens for Class I and Class II MHC molecules. The group of top-ranked neoantigens can then be further narrowed according to manufacturability and/or other criteria.

The approach begins with obtaining sequence data from the tumor. The sequence data is used to obtain data representing a polypeptide sequence of one or more tumor-specific neoantigens. The sequence data may be nucleotide sequence data, polypeptide sequence data, exome sequence data, transcriptome sequence data, or whole genome nucleotide sequence data. Suitable tumor-specific neoantigens are neoantigens that are likely presented on the cell surface of a tumor, are likely to be immunogenic, are predicted to be expressed in sufficient amounts to elicit an immune response in the subject, optionally represent sufficient diversity across the tumor, and have relatively high manufacture feasibility. The present methods take a set of neoantigens (peptide vaccine candidates) and rank the neoantigens in a way such that a group of top-ranked neoantigens simultaneously promotes cell-surface presentation of important neoantigens for Class I and Class II MHC molecules. The group of top-ranked neoantigens can then be further narrowed according to manufacturability and/or other criteria. As will be described in further detail below, the ranking is largely based on a calculated immunogenicity of the neoantigens. For short neoantigens, immunogenicity of a short neoantigen is determined at least in part based on a probability that at least one allele in a plurality of HLA class I alleles of the subject presents a short neoantigen and does not present a germline sibling of the short neoantigen. Similarly, for long neoantigens, immunogenicity of a long neoantigen is determined at least in part based on a probability that at least one allele in a plurality of HLA class II alleles of the subject presents a long neoantigen and does not present a germline sibling of the long neoantigen. These probabilities are determined using outputs provided by one or more machine-learning platforms/models, in which the machine-learning platforms/models are trained to determine a probability that a given allele presents a certain antigen.

An immunogenic composition formulated based at least in part on the present techniques may include at least about 10 tumor-specific neoantigens or at least about 20 tumor-specific neoantigens. The tumor-specific neoantigens can be encoded by short polypeptides or by long polypeptides. The immunogenic composition may comprise a nucleotide sequence, a polypeptide sequence, RNA, DNA, a cell, a plasmid, a vector, a dendritic cell, or a synthetic long peptide. The immunogenic composition can further comprise an adjuvant.

This disclosure also relates to methods of treating cancer in a subject in need thereof comprising administering a personalized immunogenic composition comprising one or more tumor specific neoantigens selected using the methods described herein. The methods disclosed herein can be suited for treating any number of cancers. The tumor can be from melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer. Preferably, the cancer is melanoma, breast cancer, lung cancer, and bladder cancer.

### BRIEF DESCRIPTION OF THE DRAWINGS

Various embodiments in accordance with the present disclosure will be described with reference to the drawings, in which:

FIG. 1 illustrates an example provider network (or “service provider system”) environment according to some embodiments.

FIG. 2 is a block diagram of an example provider network that provides a storage service and a hardware virtualization service to customers, according to some embodiments.

FIG. 3 illustrates a system that implements a portion or all of the techniques described herein, according to some embodiments.

FIG. 4 illustrates a method for ranking tumor-specific neoantigens from a tumor of a subject for a subject-specific immunogenic composition, according to an exemplary embodiment.

### DETAILED DESCRIPTION

This disclosure relates to a novel approach for ranking tumor-specific neoantigens for inclusion in potent personalized cancer immunogenic compositions (e.g., subject-specific immunogenic compositions). The disclosure also relates to methods of treating cancer in a subject in need thereof by administering an immunogenic composition comprising tumor-specific neoantigens formed using the novel approach for ranking tumor-specific neoantigens and formulating an immunogenic composition comprising the selected tumor-specific neoantigens.

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “cancer” refers to the physiological condition in subjects in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such a leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer, hormone receptor positive breast cancer), osteosarcoma, melanoma, colon cancer, colorectal cancer, endometrial (e.g., serous) or uterine cancer, salivary gland carcinoma, kidney cancer, liver cancer, prostate cancer, vulvar cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu. Hormone receptor positive breast cancer refers to breast cancer that is positive for at least one of the following: ER or PR, and negative for Her2/neu (HER2).

The term “neoantigen” as used herein refers to an antigen that has at least one alteration that makes it distinct from the corresponding parent antigen, e.g., via mutation in a tumor cell or post-translational modification specific to a tumor cell. A mutation can include a frameshift, indel, missense or nonsense substitution, splice site alteration, genomic rearrangement or gene fusion, or any genomic expression alteration giving rise to a neoantigen. A mutation can include a splice mutation. Post-translational modifications specific to a tumor cell can include aberrant phosphorylation. Post-translational modifications specific to a tumor cell can also include a proteasome-generated spliced antigen. See, Lipe et al., Science, 354(6310):354:358 (2016). In general, point mutations account for about 95% mutations in tumors and indels and frame-shift mutations account for the rest. See, Snyder et al., N Engl J Med., 371:2189-2199 (2014).

As used herein the term “tumor-specific neoantigen” is a neoantigen present in a subject's tumor cell or tissue, but not in the subject's normal cell or tissue.

The term “germline sibling” as used herein refers to germline antigens that represent the un-mutated peptide equivalent of a corresponding neoantigen.

The term “next generation sequencing” or “NGS” as used herein refers to sequencing technologies having increased throughput as compared to traditional approaches (e.g., Sanger sequencing), with the ability to generate hundreds of thousands of sequence reads at a time.

The term “neural network” as used herein refers to a machine-learning model for classification or regression consisting of multiple layers of linear transformations followed by element-wise nonlinearities typically trained via stochastic gradient descent and back-propagation.

The term “subject” as used herein refers to any animal, such as any mammal, including but not limited to, humans, non-human primates, rodents, and the like. In some embodiments, the mammal is a mouse. In some embodiments, the mammal is a human.

The term “tumor cell” as used herein refers to any cell that is a cancer cell or is derived from a cancer cell. The term “tumor cell” can also refer to a cell that exhibits cancer-like properties, e.g., uncontrollable reproduction, resistance to anti-growth signals, ability to metastasize, and loss of ability to undergo programed cell death.

Additional description of the methods and guidance for the practice of the methods are provided herein.

I. Methods for Ranking Tumor-Specific Neoantigens

Disclosed herein are methods for ranking tumor-specific neoantigens from a tumor of a subject that are suitable for subject-specific immunogenic compositions. Suitable tumor-specific neoantigens are tumor-specific neoantigens that are likely presented on the cell surface of a tumor, are likely to be immunogenic, are predicted to be expressed in sufficient amounts to elicit an immune response in the subject, optionally represent sufficient diversity across the tumor, and have relatively high manufacture feasibility. The present methods take a set of neoantigens (peptide vaccine candidates) and rank the neoantigens in a way such that a group of top-ranked neoantigens simultaneously promotes cell-surface presentation of important neoantigens for Class I and Class II MHC molecules. The group of top-ranked neoantigens can then be further narrowed according to manufacturability and/or other criteria.

Ranking the tumor-specific neoantigens from a tumor of a subject utilizes sequence data of the tumor and the subject. The sequence data of the tumor is used to obtain data representing a polypeptide sequence of one or more tumor-specific neoantigens. Generally, sequence data representing a polypeptide sequence of one or more tumor-specific neoantigens is determined by subjecting a tumor sample to sequence analysis. In some embodiments, obtaining sequence data includes receiving or accessing stored data from a previously performed sequencing. The sequence data can be, for example, exome sequence data, transcriptome sequence data, whole genome nucleotide sequence data, nucleotide sequence data, or polypeptide sequence data. Various methods of obtaining sequence data for the tumor and the subject may be used in the methods described herein. Some exemplary sequencing methods are described in further detail below.

Once sequence data representing the polypeptide sequence of one or more tumor specific neoantigens is obtained, the sequence data, along with the MHC molecule of the subject, can be analyzed in conjunction to identify and rank neoantigen candidates for inclusion in an immunogenic composition for the subject.

In one embodiment, given the set of HLA I and HLA II alleles of the subject and a list of somatic mutations of a tumor, a top-ranked set of about 30 long peptide candidates and about 15 short peptide candidates are identified and undergo manufacturability analysis. The starting set of peptides are identified using a sliding window spanning each somatic mutation. They are scored using the MHC Class I and Class II machine learning models described below. The 15 short peptides and 30 long peptides contain at least 1 MHC Class I epitope and the long peptides may also contain 1 or more MHC Class II epitopes. Then, 9 of the 30 long peptide candidates and 10 of the 15 short peptide candidates are selected for inclusion in an immunogenic composition based on manufacturability. In other embodiments, a different number of top-ranked long and/or short peptide candidates may be provided for manufacturability analysis. In some embodiments, 20-100 (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100) candidates may be provided. In other embodiment, more or fewer top-ranked candidates may be provided for manufacturability analysis.

Similarly, a different number of long and/or short peptide candidates may be ultimately selected for inclusion in the immunogenic composition. Longer neoantigens typically have more limiting manufacturing constraints than short neoantigens, thus motivating the need for a higher number of long neoantigens. In some embodiments, a neoantigen having about 15-30 amino acids (e.g., 15, 16, 17, 18, 19, 20, 21, 22, 23, 24, 25, 26, 27, 28, 29, or 30 amino acids) is considered a long neoantigen and a neoantigen having about 8-11 amino acids (e.g., 8, 9, 10, or 11 amino acids) is considered a short neoantigen. Different embodiments/implementations of the present techniques may define long and short neoantigens with different numbers of amino acids.

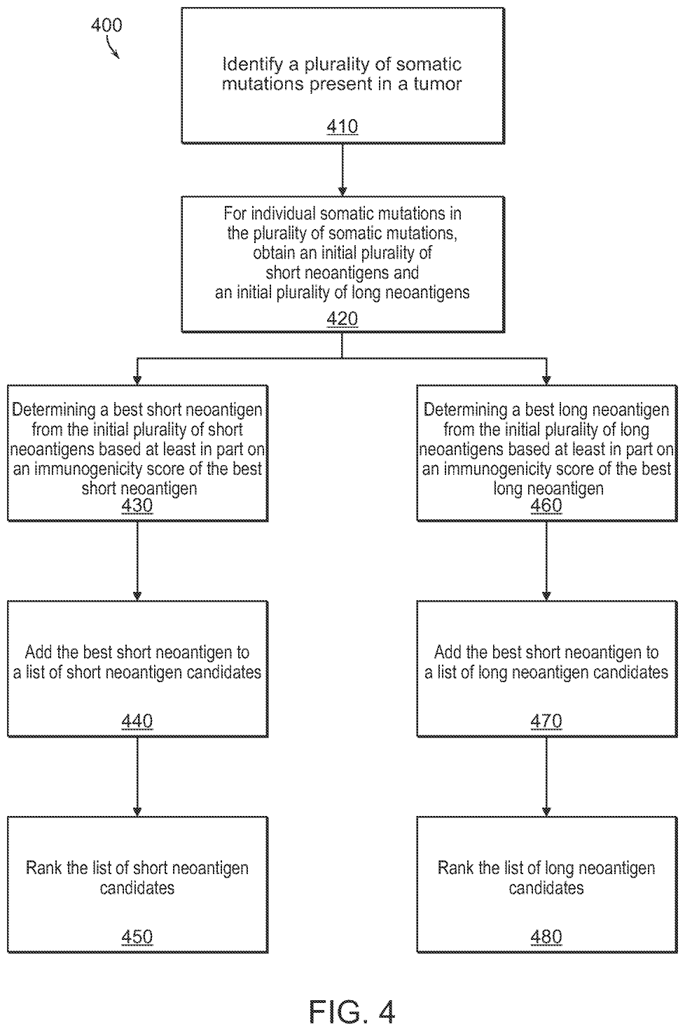

FIG. 4 illustrates an example method 400 for ranking tumor-specific neoantigens from a tumor of a subject for a subject-specific immunogenic composition. First, a plurality of somatic mutations present in the tumor are identified 410. Then, for an individual somatic mutation, an initial plurality of short neoantigens and an initial plurality of long neoantigens associated with the somatic mutation are identified or otherwise obtained 420. The initial plurality of short neoantigens can comprise short polypeptides that include at least one MHC Class I epitope associated with the subject. The initial plurality of long neoantigens can comprise long polypeptides that include at least one MHC Class I epitope and at least one MHC Class II epitope associated with the subject.

The short neoantigen in the initial plurality of short neoantigens that has the highest immunogenicity score can be selected or determined 430 and added to a list of short neoantigen candidates 440. This selected short neoantigen can also be referred to as the best short neoantigen with respect to the specified somatic mutation. Similarly, the long neoantigen in the initial plurality of long neoantigens that has the highest immunogenicity score can be selected or determined 460 and added to a list of long neoantigen candidates 470. This selected long neoantigen may also be referred to as the best long neoantigen for the specified somatic mutation. Immunogenicity scores may be any form of rating or value, numerical or non-numerical, used to represent a quality of the neoantigen with respect to one or more criteria and based upon one or more pieces of data. The immunogenicity scores of the neoantigens can be determined according to techniques described in detail below. The steps of selecting a best short neoantigen and a best long neoantigen can be performed for each somatic mutation in the plurality of somatic mutations, such that the list of short neoantigen candidates when completed includes the respective best short neoantigens for all of the somatic mutations, and wherein the list of long neoantigen candidates when completed includes the respective best long neoantigens for all of the somatic mutations. In other words, each short neoantigen in the list of short neoantigen candidates can be the best short neoantigen for a unique somatic mutation of the plurality of identified somatic mutations. Similarly, each long neoantigen in the list of long neoantigen candidates can be the best long neoantigen for a unique somatic mutation of the plurality of identified somatic mutations. The best short neoantigen and the best long neoantigen are identified for each somatic mutation. The list of short neoantigen candidates and the list of long neoantigen candidates are then each sorted and ranked 450, 480 by descending immunogenicity score.

In some embodiments, the sorted list of long neoantigen candidates are then trimmed to a predetermined number of top-ranked long neoantigen candidates. For example, the list may be trimmed to the top 30 long neoantigen candidates. Alternatively expressed, a predetermined number of top-ranked long neoantigens in the sorted list are selected for manufacturability analysis or determination. In some embodiments, the trimmed list of long neoantigen candidates (i.e., predetermined number of top-ranking long neoantigens) is provided to a manufacturer to judge manufacturability. Manufacturability of a certain neoantigen may be expressed as a numerical or non-numerical score, value, classification, or the like. Manufacturability may be based on one or a plurality of criteria or data that can be calculated, weighted, or otherwise processed in various ways. The manufacturability determination may be based on analysis performed on the actual neoantigen or based on reference materials. The manufacturer then selects a subset of long neoantigen candidates from the trimmed list of long neoantigen candidates based on manufacturability (i.e., top-ranked manufacturability scores). For example, the subset may include the top 9 long neoantigens with the highest manufacturability scores. “Manufacturer” as used herein describes any entity carrying the manufacturability analysis and selecting the subset, and could be the same entity that performs the rest of the technique or a third party.

Once the subset of long neoantigen candidates based on manufacturability are obtained, any neoantigens in the list of short neoantigen candidates that are included in any of the neoantigens in the subset of long neoantigens are removed from the list of short neoantigen candidates to remove duplicates. Alternatively expressed, for each of the long neoantigens in the subset of long neoantigen candidates, the mutation(s) in them are identified and any corresponding short neoantigens are removed from the list of short neoantigens. The remaining short neoantigens in the list of short neoantigens are then trimmed to a predetermined number based on immunogenicity score. For example, the list may be trimmed to about 15 neoantigens. Manufacturability determinations are then made for these short candidates to obtain a subset of short neoantigen candidates selected for their manufacturability. The subset of short neoantigen candidates and the subset of long neoantigen candidates are used to form or generate the subject-specific immunogenic composition which may be administered to the subject.

### Obtaining the Initial Plurality of Short Neoantigens:

In order to obtain the initial defined plurality of short neoantigens for an individual somatic mutation, first, the longest neoantigen sequence, neoT, that includes a mutated amino acid is identified. The germline sibling, neoG, for this neoantigen is also identified. Then, all neoantigen sequences that include the mutation having between a minimum (e.g., 8) and maximum (e.g., 11) number of amino acids are identified using a sliding window across the longest neoantigen sequence. This results in an initial plurality of short neoantigens, neoT_1. For example, in an embodiment in which the minimum number of amino acids is 8 and the maximum number of amino acids is 11, all neoantigen sequences within the longest neoantigen, neoT, that include the mutation and are either 8, 9, 10, or 11 amino acids in lengths are identified and designated as a member of the initial plurality of short neoantigens, neoT_1. In some embodiments, for an individual allele, a1ᵢ, in a plurality of HLA class I alleles, a1, of the subject, respective neoantigen-allele scores are determined for the identified initial plurality of short neoantigens. The neoantigen-allele score for an individual neoantigen neoT_1ⱼ of the initial plurality of short neoantigens neoT_1 and the individual allele a1ᵢ is based at least in part on a probability that the individual neoantigen is presented by the individual allele and a germline sibling of the individual neoantigen is not presented by the individual allele. This probability may be expressed as:

P1_{i,j}=p(neoT_1ⱼ|a1ᵢ)×(1−p(neoG₁ⱼ|a1ᵢ)) eq. (1)

### Where:

i is the index for neoantigens,
j is the index for alleles,
neoT_1ⱼ is the j^{th} short neoantigen in the initial plurality of short neoantigens neoT_1,
neoG_1ⱼ is the germline sibling of neoT_1ⱼ,
p(neoT_1ⱼ|a1ᵢ), equivalent to p(tumor presents|a1ᵢ), is computed by the MHC Class I machine learning model using the sequence neoT_1ⱼ and the individual allele a1ᵢ, and
p(neoG_1ⱼ|a1ᵢ), equivalent to p(germline presents|a1ᵢ), is computed by the MHC Class I machine learning model using the sequence neoG_1ⱼ and allele a1ᵢ

In some embodiments, this probability can be determined based at least in part on data from an MHC Class I machine learning model trained to determine a probability that a given allele in the plurality of HLA class I alleles presents a certain antigen.

The initial plurality of short neoantigens is further filtered such that it does not include any neoantigen that is nested in, or nests another, neoantigen of the initial plurality of short neoantigens. Such filtering can be done by identifying pairs of neoantigens in which one sequence in the pair is nested within the other, and keeps the neoantigen from the pair that has a higher probability score P1_{i,j}, as calculated using eq. (1) above. The neoantigen in the pair that has the lower probability score is removed. This process can be iterated until no such pairs remain in the initial plurality of short neoantigens, resulting in the filtered initial plurality of short neoantigens, neoT_1_filt.

### Obtaining the Initial Plurality of Long Neoantigens:

Once the initial plurality of short neoantigens is defined, a short subsequence, T1, is identified from the longest neoantigen sequence, neoT. The short subsequence, T1, is identified as the shortest subsequence of the longest neoantigen sequence neoT that includes all of the neoantigens in the initial plurality of short neoantigens, neoT_1. As mentioned above, the filtered initial plurality of short neoantigens, neoT_1_filt, has no neoantigens that are included in or includes another neoantigen in the initial plurality of short neoantigens. An expanded sequence, T1_long, can then be identified. The expanded sequence, T1_long, is obtained by adding amino acids to both sides of the short subsequence, T1, according to the longest neoantigen neoT, such that there is a first maximum number of amino acids flanking each side of the mutated amino acid. For example, the first maximum number may be 29. In some embodiments, the second maximum number of amino acids may be 9-50 (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50). Thus, in this embodiment, the expanded sequence, T1_long includes the short subsequence, T1, and 29 amino acids flanking each side of the mutated amino acid.

All possible subsequences from the long subsequence of length ranging between the length of the short subsequence, [length(T1)], and a second maximum number of amino acids can be identified and designated as the initial plurality of long neoantigens, neoT_2. For example, the second maximum number may be 30. In some embodiments, the second maximum number of amino acids may be 9-50 (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50). In some embodiments, the initial plurality of long neoantigens may be filtered based on one or more manufacturability conditions.

### Obtaining Neoantigen Immunogenicity Scores:

The immunogenicity scores of an individual short neoantigen used to select and rank (i.e., sort) the short neoantigens can be determined based at least in part on a probability that at least one allele in a plurality of HLA class I alleles of the subject presents the individual short neoantigen and does not present a germline sibling of the individual short neoantigen. This probability can be expressed as:

Class1_shortscore(neoT_1ⱼ)=1−Πᵢ₌₁^{|a1|}(1−P1_{i,j}) eq. (2)

In some embodiments, the above calculated value can also be referred to as the pan-HLA I allele score for each short neoantigen in the initial plurality of short neoantigens before it is filtered for nested pairs. The values of P1_{i,j} can be obtained using eq. (1) described above. The score calculated in eq. (2) is used to derive the immunogenicity score for each short neoantigen. In some embodiments, the immunogenicity score may be the same as the score calculated in eq. (2). In some other embodiments, the score calculated in eq. (2) may be used in further calculations or processing to arrive at the immunogenicity score.

Additionally, in some embodiments, an allele score of a peptide sequence that includes the mutated amino acid and MHC Class I epitopes for an individual MHC Class I allele can be determined based on a probability that the individual allele presents at least one neoantigen in the initial plurality of short neoantigens after it has been filtered for nested pairs, and does not present a germline sibling of the at least one neoantigen. This can be expressed as:

Class1_allelescore(T1ᵢ)=1−Πⱼ₌₁^{|neoT_1_filt|}(1−P1_{i,j}) eq. (3)

The value of P1_{i,j} can be obtained using eq. (1) described above.

In some embodiments, a pan-allele HLA Class I score can be determined based at least in part on a probability that at least one allele in a plurality of HLA Class I alleles of the subject presents at least one neoantigen in the set of short neoantigens and does not present a germline sibling of the at least one neoantigen. This can be expressed as:

Class1_longscore(T1)=1−Πᵢ₌₁^{|a1|}(1−Class1_allelescore(T1ᵢ)) eq. (4)

The immunogenicity score of an individual long neoantigen used to select and rank (i.e., sort) the long neoantigens can be determined based at least in part on a probability that at least one allele in a plurality of HLA II alleles of the subject presents the individual neoantigen and does not present a germline sibling of the individual neoantigen. Each neoantigen, neoT_2ⱼ (with index j), in the initial plurality of long neoantigens, neoT_2, is scored based on the probability that it is presented by a certain HLA II allele, allele a2ᵢ and its germline sibling neoG_2ⱼ is not, using the MHC Class II machine learning model. This probability, P2_{i,j}, is computed under the approximate assumption that presentation of the peptide and its germline sibling are independent:

P2_{i,j}=p(neoT_2ⱼ|a2ᵢ)×(1−p(neoG_2ⱼ|a2ᵢ)) eq. (5)

### Where:

neoT_2ⱼ is the j^{th} short neoantigen in the set neoT_2,
neoG_2ⱼ is the germline sibling of neoT_2ⱼ,
p(neoT_2ⱼ|a2ᵢ), equivalent to p(tumor presents|a2ᵢ), is computed by the MHC Class II machine learning model using the sequence neoT_2ⱼ and allele a2ᵢ, and p(neoG_2ⱼ|a2ᵢ), equivalent to p(germline presents|a2ᵢ), is computed by the MHC Class II machine learning model using the sequence neoG_2ⱼ and allele a2ᵢ
   Thus, the probability that at least one allele in a plurality of HLA II alleles of the subject presents the individual neoantigen and does not present a germline sibling of the individual neoantigen can be expressed as:

Class2_longscore(neoT_2ⱼ)=1|Πᵢ₌₁^{|a2|}(1−P2_{i,j}) eq. (6)

The probability is determined based at least in part on data from an MHC Class II machine learning model trained to determine a probability that a given allele in the plurality of HLA II alleles presents a certain antigen.

The probability that a mutant peptide sequence will generate an immune response on one or more HLA class I alleles may be expressed as:

S=Φ[1−Π_{i∈HLA}[1−Sᵢ]] eq (7)

or equivalently,

S=Φ[1−_{i∈HLA}[1−P(I|M,G,Aᵢ)]] eq. (8)

### Where:

S is the overall cross-allele, per-peptide score,
i∈{0,1} is a binary indicator for allele-specific CD8+ T-cell immunogenicity,
M is a mutant peptide sequence,
G is a germline sibling peptide sequence, which, according to an example, may be defined as the location in the germline genome corresponding to the location of the mutant sequence in the tumor genome,
Aᵢ is the ith HLA class 1 allele,
Φ is the estimated cellular prevalence of the mutation, P(I|M,G,Aᵢ) is the probability of generating an immune response on a specific HLA allele given M,G,Aᵢ.
   A peptide that corresponds to a mutation that is more uniformly distributed throughout the entirety of a tumor may receive a higher score than a mutation that is considered to be rare to the tumor.

Short peptides can be included directly into a vaccine, and are expected to compete with endogenously expressed peptides for binding to MHC-I. Therefore, for these peptides, the score S may be adjusted to also include a predicted binding probability for the peptide to a given MHC-I molecule. The modified score for short peptides may be expressed as:

S=Φ[1−_{i∈HLA}[1−P(bind|M,Aᵢ)P(I|M,G,Aᵢ)]] eq. (9)

### Where:

P(I|M,G,Aᵢ) is the probability of generating an immune response on a specific HLA allele given M,G,Aᵢ, and
P(bind|M,Aᵢ) is the predicted binding probability for the mutant peptide on the HLA-I allele Aᵢ.
   This score may be provided by a Class-I machine learning model, after calibrating to binding affinity data.

Allele-specific CD8+ immunogenicity may be expressed as:

P(I|M,G,Aᵢ)=P(I|M,Aᵢ)ƒ(D_{M,G})h(r){1−P(p|Aᵢ,G)[1−P(not at anchor)]} eq. (10)

### Where:

P(I|M,Aᵢ) is a germline-independent probability of immunogenicity, and
D_{M,G} is the distance to self (“DistToSelf”) between the mutant and germline sequences.

DistToSelf may be expressed as:

D_{M,G}=Σ_{i=1,i∉A}^{L}[b(Gᵢ,Gᵢ)−b(Mᵢ,Gᵢ)] eq. (11)

### Where:

L is the length of the germline or mutant sequence, whichever is longer,
The summation is taken over all indices i that are not the N-terminus and C-terminus anchor positions, also excluding any middle anchor positions and their neighbor for some HLA class I alleles,
Gᵢ and Mᵢ are the ith amino acids in the germline and mutant sequences, respectively,
b(A,B) is the entry of a matrix corresponding to amino acid A and B.

To suppress the immunogenicity probability for mutant peptides that are closer to germline peptides, the following function may be used:

ƒ(D_{M,G})≡F(D_{M,G})/F(14) eq. (12)

### Where:

$$F ( {D}_{M , G} ) = \frac{α}{1 + {e}^{- β {D}_{M , G} + γ}} + δ \qquad eq . ( 13 )$$

### Where:

α, β, γ, and δ are determined assuming that the number k of immunogenic peptides from a set of N peptides at a given integer value of D_{M,G} is binomially distributed with probability p=ƒ(D_{M,G}), and then performing a maximum likelihood estimate of p on immunogenicity data. In this example, mutant peptides that are chemically dissimilar to the germline peptide can be scored higher, and mutant peptides that are chemically similar to the germline peptide can be scored lower. This example method can be used to scale the ranked results.

In some embodiments, a method for ranking tumor-specific neoantigens from a tumor of a subject for a subject-specific immunogenic composition includes identifying a plurality of somatic mutations present in the tumor, and for each somatic mutation in the plurality of somatic mutations: determining a best short neoantigen from an initial plurality of short neoantigens based at least in part on a quality score of the best short neoantigen, and determining a best long neoantigen from an initial plurality of long neoantigens based at least in part on a quality score of the best long neoantigen. The best short neoantigen for each somatic mutation is added to a list of short neoantigen candidates and the best long neoantigen for each somatic mutation is added to a list of long neoantigen candidates. The lists are then each ranked by descending quality score. In some embodiments, the quality score is based at least in part on at least one of predicted presentation probability, predicted binding affinity, and predicted immunogenic response. In some embodiments, the quality score is based at least in part on predicted presentation probability. In some embodiments, the quality score is based at least in part on predicted binding affinity. In some embodiments, the predicted binding affinity is determined based at least in part on data from an MHC Class II learning model trained to determine the binding affinity between a Class II allele and a given peptide. In some embodiments, the quality score is based at least in part on predicted immunogenic response. In some embodiments, the quality score is based at least in part on a combination of predicted presentation probability, predicted binding affinity, and predicted presentation probability. In some embodiments, the predicted presentation probability, predicted binding affinity, and predicted presentation probability are determined by one or more machine learning models.

In some embodiments, the peptides may be filtered for consideration or inclusion in the final subject-specific immunogenic composition based on any subset of the following criteria: 1) RNA abundance (measured in transcripts per million, TPM) for the gene to which the somatic mutation belongs. For example, RNA abundance may be determined by multiplying the RNA TPM value of a gene to which the variant belongs by a ratio of the number of reads overlapping the variant locus that contain the variant allele to the sum of (a) the number of reads overlapping the variant locus that contain the variant allele and (b) the total number of reads overlapping the variant locus. 2) Whether the somatic mutation is in an essential gene or driver gene. Driver genes are genes whose mutations can cause tumor growth. Essential genes are genes that are critical for the survival of the organism. 3) Whether the peptides are predicted to pass quality control thresholds on synthesizability and solubility. 4) How foreign (i.e., different) a mutated peptide is from the corresponding germline peptide. In some embodiments, a minimum number of mutated amino acids may be required for the peptide to be considered or included, and priority may be given to highly foreign peptides over less foreign peptides. 5) Confidence level that a particular mutation is present in the particular subject. For example, rare somatic mutations are given lower confidence scores than more frequently occurring mutations. 6) Whether a peptide candidate includes certain amino acids, such as cysteine.

A somatic variant can mutate zero, one, or multiple amino acids. For example, silent mutations mutate zero amino acids, single nucleotide variants typically mutate one amino acid, and frame-shift or stop-loss mutations can mutate multiple amino acids. If RNA super reads are found assembled upstream at the variant locus, the longest consensus mRNA sequence that overlaps with mutant amino acids will be assembled. The mRNA sequence assembly will stop if RNA read coverage ends or if a new stop codon is found. If no RNA super reads are found, the mRNA sequence assembly will stop when no mutant amino acids are found past the requested protein sequence length.

Predicted presentation data may consist entirely of “positive” samples, which can be presented on the cell-surface. Therefore, to train such a predictor, which may require “negative” samples that cannot be presented on the cell-surface, one or more probabilistic negative mining strategies may be employed during training. Such processes may include HLA allele shuffling, where when given a positive sample (e.g., a peptide and corresponding HLA allele), the given allele can be replaced by randomly sampling a different allele that does not belong to the positive allele's supertype(s). Each HLA allele may be classified to one or more HLA supertypes, until only unclassified HLA alleles remain. The unclassified HLA alleles may be mapped to one or more “unclassified” supertype classes, and these groups may be processed similarly to the classified supertype classes.

Additionally, peptide shuffling may be employed to train the predictor where, given a positive sample consisting of a peptide and corresponding HLA allele, the given peptide is replaced with a randomly-sampled amino acid subsequence, of the same length, from the peptide's source protein.

Random peptides may also be generated, to help train the predictor. According to this example, random peptides, sampled from amino-acid data distribution, can be generated, with qualitative affinity targets falling below a determined threshold and negative presentation targets. The length of the random peptides may be determined such that an equal number of non-binding data points exists, per peptide length, for each allele. A determined ratio (e.g., 10:1) between negative and positive presenting samples may be sampled, and a sample weight may be applied to the negative samples for a balanced loss. For each negative sample, the sampling method can be chosen randomly with uniform distribution.

### Sequencing Methods

Various sequencing methods are well known in the art and include, but are not limited to, PCR-based methods, including real-time PC, whole exome sequencing, deep sequencing, high-throughput sequencing, or combinations thereof. In some embodiments, the foregoing techniques and procedures are performed according to the methods described in e.g., Sambrook et al., Molecular Cloning: A Laboratory Manual 4th ed. (2012) Cold Spring Harbor Laboratory Press, Cold Spring Harbor, N.Y. See also, Austell et al., Current Protocols in Molecular Biology, ed., Greene Publishing and Wiley-Interscience New York (1992) (with periodic updates).

Sequencing methods may also include, but are not limited to, high-throughput sequencing, single-cell RNA sequence, RNA sequencing, pyrosequencing, sequencing-by synthesis, single-molecule sequencing, nanopore sequencing, semiconductor sequencing, sequencing-by-synthesis, sequencing-by-ligation, sequencing-by-hybridization, RNA-Sew (Illumina), Digital Gene Expression (Helicos), next generation sequencing, Single Molecule Sequencing by Synthesis (SMSS) (Helicos), massively-parallel sequencing, Clonal Single Molecule Array (Solexa), shotgun sequencing, Maxam-Hilbery or Sanger sequencing, whole genome sequencing, whole exome sequencing, primer walking, sequencing using PacBio, SOLid, Ion Torrent, or Nanopore platforms and any other sequencing methods known in the art. The sequencing method employed herein to obtain sequence data is preferably high-throughput sequencing. High-throughput sequencing technologies are capable of sequencing multiple nucleic acid molecules in parallel, enabling millions of nucleic acid molecules to be sequenced at a time. See, Churko et al., Circ. Res. 112(12):1613-1623 (2013).

In some cases, high-throughput sequencing can be next generation sequencing. There are a number of different next generation platforms using different sequencing technologies (e.g., using the HiSeq or MiSeq instruments available from Illumina (San Diego, Calif.)). Any of these platforms can be employed for sequencing the genetic material disclosed herein. Next generation sequencing is based on sequencing a large number of independent reads, each representing anywhere between 10 to 1000 bases of nucleic acid. Sequencing by synthesis is a common technique used in next generation sequencing. In general, sequencing involves hybridizing a primer to a template to form a template/primer duplex, contacting the duplex with a polymerase in the presence of a detectably-labeled nucleotide under conditions that permit the polymerase to add nucleotides to the primer in a template-dependent manner. Signal from the detectable label is then used to identify the incorporated base and the steps are sequentially repeated in order to determine the linear order of nucleotides in the template. Exemplary detectable labels include radiolabels, florescent labels, enzymatic labels, etc. Numerous techniques are known for detecting sequences, such as the Illumina NextSeq platform by cycle end sequencing.

### Machine-Learning Models

Once sequence data representing the polypeptide sequence of one or more tumor specific neoantigens is obtained, the sequence data, along with the MHC molecule of the subject, can be inputted into a machine-learning platform (i.e., model(s)). The machine-learning platform can generate one or more numerical probability scores that forecast whether the one or more tumor-specific neoantigens are immunogenic (e.g. will elicit an immune response in the subject.

MHC molecules transport and present peptides on the cell surface. The MHC molecules are classified as MHC molecules of Class I and of Class II. MHC Class I are present on the surface of almost all cells of the body, including most tumor cells. The proteins of MHC Class I are loaded with antigens that usually originate from endogenous proteins or from pathogens present inside cells, and are then presented to cytotoxic T-lymphocytes (i.e., CD8+). The MHC Class I molecules can comprise HLA-A, HLA-B, or HLA-C. The MHC molecules of Class II are only present on dendritic cells, B lymphocytes, macrophages and other antigen-presenting cells. They present mainly peptides, which are processed from external antigen sources, i.e. outside of the cells, to T-helper (Th) cells (i.e., CD4+). The MHC Class II molecules can comprise HLA-DPA1, HLA-DPB1, HLA-DQA1, HLA-DQB1, HLA-DRA, and HLA-DRB1. In some occasions, MHC Class II molecules can also be expressed on cancer cells.

MHC Class I molecules and/or MHC Class II molecules can be inputted into the machine-learning platform. Typically, either MHC Class I molecules or MHC Class II molecules are inputted into the machine-learning platform. In some embodiments, MHC Class I molecules are inputted into the machine-learning platform. In other embodiments, MHC Class II molecules are inputted into the machine-learning platform. In some embodiments, an MHC Class I machine-learning platform may be trained on MHC Class I training data. In some embodiments, an MHC Class II machine-learning platform may be trained on MHC Class II training data. In some embodiments the same machine-learning platform may be trained on both MHC Class I and Class II training data. In some embodiments, the machine-learning platform may include an MHC Class I model and an MHC Class II mode.

MHC Class I molecules bind to short peptides. MHC Class I molecules can accommodate peptides generally about 8 amino acids to about 10 amino acids in length. In embodiments, the sequence data encoding one or more tumor-specific neoantigens are short peptides about 8 amino acids to about 10 amino acids in length. MHC Class II molecules bind to peptides that are longer in length. MHC Class II can accommodate peptides which are generally about 13 amino acids in length to about 25 amino acids in length. In embodiments, the sequence data encoding one or more tumor-specific neoantigens are long peptides about 13 to 25 amino acids in length.

The sequence data encoding one or more tumor-specific neoantigens can be about 5 amino acids in length, about 6 amino acids in length, about 7 amino acids in length, about 8 amino acids in length, about 9 amino acids in length, about 10 amino acids in length, about 11 amino acids in length, about 12 amino acids in length, about 13 amino acids in length, about 14 amino acids in length, about 15 amino acids in length, about 16 amino acids in length, about 17 amino acids in length, about 18 amino acids in length, about 19 amino acids in length, about 20 amino acids in length, about 21 amino acids in length, about 22 amino acids in length, about 23 amino acids in length, about 24 amino acids in length, about 25 amino acids in length, about 26 amino acids in length, about 27 amino acids in length, about 28 amino acids in length, about 29 amino acids in length, or about 30 amino acids in length.

The machine-learning platform can predict the likelihood that one or more tumor-specific neoantigens are immunogenic (e.g., will elicit an immune response).

Immunogenic tumor-specific neoantigens are not expressed in normal tissues. They can be presented by antigen-presenting cells to CD4+ and CD8+ T-cells to generate an immune response. In embodiments, an immune response in the subject elicited by the one or more tumor-specific neoantigens comprises presentation of the one or more tumor-specific neoantigens to the tumor cell surface. More specifically, the immune response in the subject elicited by the one or more tumor-specific neoantigens comprises presentation of the one or more tumor-specific neoantigens by one or more MHC molecules on the tumor cell. It is expected that the immune response elicited by the one or more tumor-specific neoantigens is a T-cell mediated response. The immune response in the subject elicited by the one or more tumor-specific neoantigens may involve one or more tumor-specific neoantigens being capable of presentation to T-cells by antigen presenting cells, such as dendritic cells. Preferably, the one or more tumor-specific neoantigens is capable of activating CD8+ T-cells and/or CD4+ T-cells.

In some embodiments, the machine-learning platform can predict the likelihood the one or more tumor-specific neoantigens will activate CD8+ T cells. In embodiments, the machine learning platform can predict the likelihood that the one or more tumor-specific neoantigens will activate CD4+ T cells. In some instances, the machine-learning platform can predict the antibody titer that the one or more tumor-specific neoantigens can elicit. In other instances, the machine-learning platform can predict the frequency of CD8+ activation by the one or more tumor-specific neoantigens.

The machine-learning platform can include a model trained on training data. Training data can be obtained from a series of distinct subjects. The training data can comprise data derived from healthy subjects, as well as subjects having cancer. The training data may include various data that can be used to generate a probability score that indicates whether the one or more tumor-specific neoantigens will elicit an immune response in a subject. Exemplary training data can include data representing nucleotide or polypeptide sequences derived from normal tissue and/or cells, data representing nucleotide or polypeptide sequences derived from tumor tissue, data representing MHIC peptidome sequences from normal and tumor tissue, peptide-MHIC binding affinity measurement, or combinations thereof. The reference data can further comprise mass spectrometry data, DNA sequencing data, RNA sequencing data, clinical data from healthy subjects and subjects having cancer, cytokine profiling data, T cell cytotoxicity assay data, peptide-MHIC mono-or-multimer data, and proteomics data for single-allele cell lines engineered to express a predetermined MIC allele that are subsequently exposed to synthetic protein, normal and tumor human cell lines, fresh and frozen primary samples, and T-cell assays.

In some example embodiments, binding affinity predictions for various samples may be extracted and added to a binding affinity training dataset, including corresponding “weak” labels for samples that have an unknown binding affinity prediction. Samples in which the binding affinity prediction exceeds a determined threshold may be filtered out, leaving a distilled dataset for use in further training processes.

The machine-learning platform can be a supervised learning platform, an unsupervised learning platform, or a semi-supervised learning platform. The machine-learning platform can use sequence-based approach to generate a numerical probability that the one or more tumor-specific neoantigens can elicit an immune response (e.g., will induce a high or low antibody response or CD8+ response). Sequence based predictions can include supervised machine-learning modules including, artificial neural networks (e.g., deep or otherwise), support vector machines, K-nearest neighbor, Logistic Multiple Network-constrained Regression (LogMiNeR), regression tree, random forest, adaboost, XGBoost, or hidden Markov models. These platforms require training data sets that include known MHC binding peptides.

According to some embodiments, masked language modeling may be implemented in a pre-training phase, such that a determined subset of the peptide sequence may be masked via a tokenization process. A classifier may then predict the original token values, based on existing tokens that are not masked.

According to another example, a next peptide in a sequence may be determined in accordance with a pre-training process where an input sequence may be a concatenation of two peptide sequences, instead of a peptide and allele sequence in a main training phase. The two peptide sequences can be separated using a special separation token, and each segment may have a different segment index and embedding. The segment sequence may be provided as an input to the network, indicating whether each token belongs to a first sequence, a second sequence, or is a special token. A classifier can be trained, using the token, to predict whether a second peptide is the next occurring peptide in the protein. The peptides may be provided by two consecutive, same-length peptides from a human protein, or may be randomly-sampled from different proteins.

Numerous prediction programs have been employed to predict whether a tumor-specific neoantigen can be presented on an MHC molecule and elicit an immune response. Exemplary predictive programs include, for example, HLAminer (Warren et al., Genome Med., 4:95 (2012); HLA type predicted by orienting the assembly of shotgun sequence data and comparing it with the reference allele sequence database), VariantEffect Predictor Tool (McLaren et al., Genome Biol., 17:122 (2016)), NetMHCpan (Andreatta et al., Bioinformatics., 32:511-517 (2016); sequence comparison method based on artificial neural network, and predict the affinity of peptide-MHC-I type molecular), UCSC browser (Kent et al., Genome Res., 12:996-1006 (2002)), CloudNeo pipeline (Bais et al., Bioinformatics, 33:3110-2 (2017)), OptiType (Szolek et al., Bioinformatics, 30:3310-316 (2014)), ATHLATES (Liu C et al., Nucleic Acids Res. 41:e142 (2013)), pVAC-Seq (Hundal et al., Genome Med. 8:11 (2016), MuPeXI (Bjerregaard et al., Cancer Immunol Immunother., 66:1123-30 (2017)), Strelka (Saunders et al., Bioinformatics. 28:1811-7 (2012)), Strelka2 (Kim et al., Nat Methods. 2018; 15:591-4), VarScan2 (Koboldt et al., Genome Res., 22:568-76 (2012)), Somaticseq (Fang L et al., Genome Biol., 16:197 (2015)), SMMPMBEC (Kim et al., BMC Bioinformatics., 10:394 (2009)), NeoPredPipe (Schenck R O, BMC Bioinformatics., 20:264 (2019)), Weka (Witten et al., Data mining: practical machine-learning tools and techniques. 4^{th} ed. Elsevier, ISBN: 97801280435578 (eBook) (2017), or Orange (Demsar et al., Orange: Data Mining Toolbox in Python., J. Mach Learn Res., 14:2349-2353 (2013). Any known predictive programs may be employed as the machine-learning platform to generate a numerical probability score that indicates whether the neoantigen will elicit an immune response.

Depending on the machine-learning platform employed, additional filters can be applied to prioritize tumor-specific neoantigen candidates, including: elimination of hypothetical (Riken) proteins; use of an antigen processing algorithm to eliminate epitopes that are not likely to be proteolytically produced by the constitutive- or immune-proteasome and prioritization of neoantigens where the neoantigen has a higher predicted binding affinity than the corresponding wildtype sequence.

The numerical probability score can be a number between 0 and 1. In embodiments, the numerical probability score can be a number of 0, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, or 1. A tumor-specific neoantigen with a higher numerical probability score relative to a lower numerical probability score indicates that the tumor-specific neoantigen will elicit a greater immune response in the subject, and thus is likely to be a suitable candidate for an immunogenic composition. For example, a tumor-specific neoantigen with a numerical probability score of 1 will likely elicit a greater immune response in a subject than a tumor-specific neoantigen having a numerical probability score of 0.05. Similarly, a tumor-specific neoantigen having a numerical probability score of 0.5 will likely elicit a greater immune response in a subject than a tumor-specific neoantigen with a numerical probability score of 0.1.

A higher numerical probability score relative to a lower numerical probability score is preferable. Preferably, tumor-specific neoantigen having a numerical probability score of at least 0.8, 0.81, 0.82, 0.83, 0.84, 0.85, 0.86, 0.87, 0.88, 0.89, 0.9, 0.95, 0.96, 0.97, 0.98, 0.99, or 1 indicates that an immune response will likely be elicited in the subject.

While a higher numerical probability score is preferable, a lower numerical probability score may still indicate that the tumor-specific neoantigen is capable of eliciting a sufficient immune response, such that the tumor-specific neoantigen is likely to be a suitable candidate.

In instances, the machine-learning platform described herein can also predict the likelihood that the one or more tumor-specific neoantigens will be presented by a MHC molecule on a tumor cell. The machine-learning platform can predict the likelihood that one or more tumor-specific neoantigens will be presented by a MHC Class I molecule or MHC Class II molecule.

The methods for selecting one or more tumor-specific neoantigens may further comprise a step of measuring, in silico, the affinity of one or more tumor-specific neoantigens to bind to a MHC molecule in the subject. A tumor-specific neoantigen that has a binding affinity with a MHC molecule of less than about 1000 nM indicates that the one or more tumor-specific neoantigens may be suitable for an immunogenic composition. A tumor-specific neoantigen that has a binding affinity with a MHC molecule of less than about 500 nM, of less than about 400 nM, of less than about 300 nM, of less than about 200 nM, of less than about 100 nM, of less than about 50 nM can indicate that one or more tumor-specific neoantigens may be suitable for an immunogenic composition. The affinity of the one or more tumor-specific neoantigens to bind to a MHC molecule in the subject can predict tumor-specific neoantigen immunogenicity. Alternatively, median affinity can be an effective way to predict tumor-specific neoantigen immunogenicity. Median affinity can be calculated using epitope prediction algorithms, such as NetMHCpan, ANN, SMM and SMMPMBEC.

RNA expression of one or more tumor-specific neoantigens is also quantified. RNA expression of one or more tumor-specific neoantigens is quantified to identify one or more neoantigens that will elicit an immune response in a subject. A variety of methods exist for measuring RNA expression. Known techniques, which may measure RNA expression, include RNA-seq, and in situ hybridization (e.g., FISH), Northern blot, DNA microarray, Tiling array, and quantitative polymerase chain reaction (qPCR). Other known techniques in the art can be used to quantify RNA expression. RNA can be messenger RNA (mRNA), short-interfering RNA (siRNA), microRNA (miRNA), circular RNA (circRNA), transfer RNA (tRNA), ribosomal RNA (rRNA), small nucleolar RNA (snRNA), Piwi-interacting RNA (piRNA), long non-coding RNA (long ncRNA), sub-genomic RNA (sgRNA), RNA from integrating or non-integrating viruses, or any other RNA. Preferably, mRNA expression is measured.

The present technique can further reduce the likelihood of selecting tumor-specific neoantigen may induce an autoimmune response in normal tissues. It is expected that a tumor-specific neoantigen that has similar sequence to a normal antigen may induce an autoimmune response in normal tissue. For example, a tumor-specific neoantigen that is at least 90%, at least 91%, at least 92%, at least 93%, at least 94%, at least 95%, at least 96%, at least 97%, at least 98%, or at least 99% similar to a normal antigen may induce an autoimmune response. Tumor-specific neoantigens that are predicted to induce an autoimmune response are not prioritized for the immunogenic composition. Tumor-specific neoantigens that are predicted to induce an autoimmune response are typically not selected for the immunogenic composition. The method can further comprise measuring the ability of the one or more tumor-specific neoantigen to invoke immunological tolerance. Tumor-specific neoantigens that are predicted to invoke immunological tolerance are not prioritized for the immunogenic composition. Tumor-specific neoantigens that are predicted to invoke immunological tolerance are not prioritized for the immunogenic composition.

Finally, one or more tumor-specific neoantigens based on the tumor-specific score are selected for formulation of a subject-specific immunogenic composition. In embodiments, at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 50 or more tumor-specific neoantigens are selected for the immunogenic composition. Typically, at least about 10 tumor-specific neoantigens are selected. In other instances, at least about 20 tumor-specific neoantigens are selected.

II. Methods of Treating

This disclosure also relates to methods of treating cancer in a subject in need thereof comprising administering a personalized immunogenic composition comprising one or more tumor specific neoantigens selected using the methods described herein.

The cancer can be any solid tumor or any hematological tumor. The methods disclosed herein are preferably suited for solid tumors. The tumor can be a primary tumor (e.g., a tumor that is at the original site where the tumor first arose). Solid tumors can include, but are not limited to, breast cancer tumors, ovarian cancer tumors, prostate cancer tumors, lung cancer tumors, kidney cancer tumors, gastric cancer tumors, testicular cancer tumors, head and neck cancer tumors, pancreatic cancer tumors, brain cancer tumors, and melanoma tumors. Hematological tumors can include, but are not limited to, tumors from lymphomas (e.g., B cell lymphomas) and leukemias (e.g., acute myelogenous leukemia, chronic myelogenous leukemia, chronic lymphocytic leukemia, and T cell lymphocytic leukemia).

The methods disclosed herein can be used for any suitable cancerous tumor, including hematological malignancy, solid tumors, sarcomas, carcinomas, and other solid and non-solid tumors. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. Preferably, the cancer is melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer. Melanoma is of particular interest. Breast cancer, lung cancer, and bladder cancer are also of particular interest.

Immunogenic compositions stimulate a subject's immune system, especially the response of specific CD8+ T cells or CD4+ T cells. Interferon gamma produced by CD8+ and T helper CD4+ cells regulate the expression of PD-L1. PD-L1 expression in tumor cells is upregulated when attacked by T cells. Therefore, tumor vaccines may induce the production of specific T cells and simultaneously upregulate the expression of PD-L1, which may limit the efficacy of the immunogenic composition. In addition, while the immune system is activated, the expression of T cell surface reporter CTLA-4 is correspondingly increased, which binds with the ligand B7-1/B7-2 on antigen-presenting cells and plays an immunosuppressant effect. Thus, in some instances, the subject may further be administered an anti-immunosuppressive or immunostimulatory, such as a checkpoint inhibitor. Checkpoint inhibitors can include, but are not limited to, anti-CTL4-A antibodies, anti-PD-1 antibodies and anti-PD-L1 antibodies. These checkpoint inhibitors bind to the immune checkpoint proteins of T cells to remove the inhibition of T cell function by tumor cells. Blockade of CTLA-4 or PD-L1 by antibodies can enhance the immune response to cancerous cells in the patient. CTLA-4 has been shown effective when following a vaccination protocol.

An immunogenic composition comprising one or more tumor-specific neoantigens can be administered to a subject that has been diagnosed with cancer, is already suffering from cancer, has recurrent cancer (i.e., relapse), or is at risk of developing cancer. An immunogenic composition comprising one or more tumor-specific neoantigens can be administered to a subject that is resistant to other forms of cancer treatment (e.g., chemotherapy, immunotherapy, or radiation). An immunogenic composition comprising one or more tumor-specific neoantigens can be administered to the subject prior to other standard of care cancer therapies (e.g., chemotherapy, immunotherapy, or radiation). An immunogenic composition comprising one or more tumor-specific neoantigens can be administered to the subject concurrently, after, or in combination to other standard of care cancer therapies (e.g., chemotherapy, immunotherapy, or radiation).

The subject can be a human, dog, cat, horse, or any animal for which a tumor specific response is desired.

The immunogenic composition is administered to the subject in an amount sufficient to elicit an immune response to the tumor-specific neoantigen and to destroy, or at least partially arrest, symptoms and/or complications. In embodiments, the immunogenic composition can provide a long-lasting immune response. A long-lasting immune response can be established by administering a boosting dose of the immunogenic composition to the subject. The immune response to the immunogenic composition can be extended by administering to the subject a boosting dose. In embodiments, at least one, at least two, at least three or more boosting doses can be administered to abate the cancer. A first boosting dose may increase the immune response by at least 50%, at least 100%, at least 200%, at least 300%, at least 400%, at least 500%, or at least 1000%. A second boosting dose may increase the immune response by at least 50%, at least 100%, at least 200%, at least 300%, at least 400%, at least 500%, or at least 1000%. A third boosting dose may increase the immune response by at least 50%, at least 100%, at least 200%, at least 300%, at least 400%, at least 500%, or at least 1000%.

An amount adequate to elicit an immune response is defined as a “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that immunogenic compositions can generally be employed in serious disease states, that is, life-threatening or potentially life-threatening situations, especially when the cancer has metastasized. In such cases, in view of the minimization of extraneous substances and the relative nontoxic nature of a neoantigen, it is possible and can be felt desirable by the treating physician to administer substantial excesses of these immunogenic compositions.

The immunogenic composition comprising one or more tumor-specific neoantigens can be administered to the subject alone or in combination with other therapeutic agents. The therapeutic agent can be, for example, a chemotherapeutic agent, radiation, or immunotherapy. Any suitable therapeutic treatment for a particular cancer can be administered. Exemplary chemotherapeutic agents include, but are not limited to aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. The subject may be administered a small molecule, or targeted therapy (e.g. kinase inhibitor). The subject may be further administered an anti-CTLA antibody or anti-PD-1 antibody or anti-PD-L1 antibody. Blockade of CTLA-4 or PD-L1 by antibodies can enhance the immune response to cancerous cells in the patient.

III. Immunogenic Compositions

The invention further relates to personalized (i.e., subject-specific) immunogenic compositions (e.g., a cancer vaccine) comprising one or more tumor-specific antigens selected using the methods described herein. Such immunogenic compositions can be formulated according to standard procedures in the art. The immunogenic composition is capable of raising a specific immune response.

The immunogenic composition can be formulated so that the selection and number of tumor-specific neoantigens is tailored to the subject's particular cancer. For example, the selection of the tumor-specific neoantigens can be dependent on the specific type of cancer, the status of the cancer, the immune status of the subject, and the MHC-type of the subject.

The immunogenic composition can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 37, 37, 38, 39, 40, 41, 42, 43, 44, 45, 46, 47, 48, 49, 50 or more tumor-specific neoantigens. The immunogenic composition can contain about 10-20 tumor-specific neoantigens, about 10-30 tumor-specific neoantigens, about 10-40 tumor-specific neoantigens, about 10-50 tumor-specific neoantigens, about 10-60 tumor-specific neoantigens, about 10-70 tumor-specific neoantigens, about 10-80 tumor-specific neoantigens, about 10-90 tumor-specific neoantigens, or about 10-100 tumor-specific neoantigens. Preferably, the immunogenic composition comprises at least about 10 tumor-specific neoantigens. Also preferably is an immunogenic composition that comprises at least about 20 tumor-specific neoantigens.

The immunogenic composition can further comprise natural or synthetic antigens. The natural or synthetic antigens can increase the immune response. Exemplary natural or synthetic antigens include, but are not limited to, pan-DR epitope (PADRE) and tetanus toxin antigen.

The immunogenic composition can be in any form, for example a synthetic long peptide, RNA, DNA, a cell, a dendritic cell, a nucleotide sequence, a polypeptide sequence, a plasmid, or a vector.

Tumor-specific neoantigens can also be included in viral vector-based vaccine platforms, such as vaccinia, fowlpox, self-replicating alphavims, marabavirus, adenovirus (See, e.g., Tatsis et al., Molecular Therapy, 10:616-629 (2004)), or lentivirus, including but not limited to second, third or hybrid second/third generation lentivirus and recombinant lentivirus of any generation designed to target specific cell types or receptors (See, e.g., Hu et al., Immunol Rev., 239(1): 45-61 (2011), Sakma et al, Biochem J., 443(3):603-18 (2012)). Dependent on the packaging capacity of the above-mentioned viral vector-based vaccine platforms, this approach can deliver one or more nucleotide sequences that encode one or more tumor-specific neoantigen peptides. The sequences may be flanked by non-mutated sequences, may be separated by linkers or may be preceded with one or more sequences targeting a subcellular compartment (See, e.g., Gros et al., Nat Med., 22 (4):433-8 (2016), Stronen et al., Science., 352(6291): 1337-1341 (2016), Lu et al., Clin Cancer Res., 20(13):3401-3410 (2014)). Upon introduction into a host, infected cells express the one or more tumor-specific neoantigens, and thereby elicit a host immune (e.g., CD8+ or CD4+) response against the one or more tumor-specific neoantigens. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of neoantigens that will be apparent to those skilled in the art from the description herein may also be used.

The immunogenic composition can contain individualized components, according to their personal needs of the particular subject.

The immunogenic composition described herein can further comprise an adjuvant. Adjuvants are any substance whose admixture into an immunogenic composition increases, or otherwise enhances and/or boosts, the immune response to a tumor-specific neoantigen, but when the substance is administered alone does not generate an immune response to a tumor-specific neoantigen. The adjuvant preferably generates an immune response to the neoantigen and does not produce an allergy or other adverse reaction. It is contemplated herein that the immunogenic composition can be administered before, together, concomitantly with, or after administration of the immunogenic composition.

Adjuvants can enhance an immune response by several mechanisms including, e.g., lymphocyte recruitment, stimulation of B and/or T cells, and stimulation of macrophages. When an immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see, GB 2220211), MF59 (Novartis), ASO3 (Glaxo SmithKline), AS04 (Glaxo SmithKline), polysorbate 80 (Tween 80; ICL Americas, Inc.), imidazopyridine compounds (see, International Application No. PCT/US2007/064857, published as International Publication No. WO2007/109812), imidazoquinoxaline compounds (see, International Application No. PCT/US2007/064858, published as International Publication No. WO2007/109813) and saponins, such as QS21 (see, Kensil et al, in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see, Stoute et al, N. Engl. J. Med. 336, 86-91 (1997)).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), poly ICLC, non-CpG bacterial DNA or RNA as well as immunoactive small molecules and antibodies such as cyclophosphamide, sunitmib, bevacizumab, Celebrex (celecoxib), NCX-4016, sildenafil, tadalafil, vardenafil, sorafinib, XL-999, CP-547632, pazopamb, ZD2171, AZD2171, ipilimumab, tremelimumab, and SC58175, which may act therapeutically and/or as an adjuvant. In embodiments, Poly ICLC is a preferable adjuvant.

The immunogenic compositions can comprise one or more tumor-specific neoantigens described herein alone or together with a pharmaceutically acceptable carrier. Suspensions or dispersions of one or more tumor-specific neoantigens, especially isotonic aqueous suspensions, dispersions, or ampgipgilic solvents can be used. The immunogenic compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

An excipient can be present independently of an adjuvant. The function of an excipient can be, for example, to increase the molecular weight of the immunogenic composition, to increase activity or immunogenicity, to confer stability, to increase the biological activity, or to increase serum-half life. An excipient can also be used to aid presentation of the one or more tumor-specific neoantigens to T-cells (e.g., CD 4+ or CD8+ T-cells). The excipient can be a carrier protein such as, but not limited to, keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. Alternatively, the carrier can be dextran, for example sepharose.

Cytotoxic T-cells recognizes an antigen in the form of a peptide bound to an MHC molecule, rather than the intact foreign antigen itself. The MHC molecule itself is located at the cell surface of an antigen presenting cell. Thus, an activation of cytotoxic T-cells is possible if a trimeric complex of peptide antigen, MHC molecule, and antigen-presenting cell (APC) is present. It may enhance the immune response if not only the one or more tumor-specific antigens are used for activation of cytotoxic T-cells, but if additional APCs with the respective MHC molecule are added. Therefore, in some embodiments an immunogenic composition additionally contains at least one APC.

The immunogenic composition can comprise an acceptable carrier (e.g., an aqueous carrier). A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Neoantigens can also be administered via liposomes, which target them to a particular cell tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the neoantigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired neoantigen can be directed to the site of lymphoid cells, where the liposomes then deliver the selected immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., An. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

An alternative method for targeting immune cells, components of the immunogenic composition, such as an antigen (i.e., tumor-specific neoantigen), ligand, or adjuvant (e.g., TLR) can be incorporated into an poly(lactic-co-glycolic) microspheres. The poly(lactic-co-glycolic) microspheres can entrap components of the immunogenic composition as an endosomal delivery device.

For therapeutic or immunization purposes, nucleic acids encoding a tumor-specific neoantigen described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990), as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation. The nucleic acids can also be delivered complexed to cationic compounds, such as cationic lipids.

The immunogenic compositions provided herein can be administered to the subject by, including but not limited to, oral, intradermal, intratumoral, intramuscular, intraperitoneal, intravenous, topical, subcutaneous, percutaneous, intranasal and inhalation routes, and via scarification (scratching through the top layers of skin, e.g., using a bifurcated needle). The immunogenic composition can be administered at the tumor site to induce a local immune response to the tumor.

The dosage of the one or more tumor-specific neoantigens may depend upon the type of composition and upon the subject's age, weight, body surface area, individual condition, the individual pharmacokinetic data, and the mode of administration.

Also disclosed herein is a method of manufacturing an immunogenic composition comprising one or more tumor-specific neoantigens selected by performing the steps of the methods disclosed herein. An immunogenic composition as described herein can be manufactured using methods known in the art. For example, a method of producing a tumor-specific neoantigen or a vector (e.g., a vector including at least one sequence encoding one or more tumor-specific neoantigens) disclosed herein can include culturing a host cell under conditions suitable for expressing the neoantigen or vector, wherein the host cell comprises at least one polynucleotide encoding the neoantigen or vector, and purifying the neoantigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes one or more tumor-specific neoantigens or vector disclosed herein. In certain embodiments the isolated polynucleotide can be cDNA.

IV. Samples

The methods disclosed herein comprise ranking one or more tumor-specific neoantigens derived from a tumor. The methods of ranking one or more tumor-specific neoantigens comprise obtaining sequence data derived from the tumor. Such sequence data can be derived from a tumor sample of a subject. The tumor sample can be obtained from a tumor biopsy.

The tumor sample can be obtained from human or non-human subjects. Preferentially, the tumor sample is obtained from a human. The tumor sample can be obtained from a variety of biological sources that comprise cancerous tumors. The tumor can be from a tumor site or circulating tumor cells from blood. Exemplary samples can include, but are not limited to, bodily fluid, tissue biopsies, blood samples, serum plasma, stool, skin samples, and the like. The source of a sample can be a solid tissue sample such as a tumor tissue biopsy. Tissue biopsy samples may be biopsies from, e.g., lung, prostate, colon, skin, breast tissue, or lymph nodes. Samples can also be e.g., samples of bone marrow, including bone marrow aspirate and bone marrow biopsies. Samples can also be liquid biopsies, e.g., circulating tumor cells, cell-free circulating tumor DNA, or exosomes. Blood samples can be whole blood, partially purified blood, or a fraction of whole or partially purified blood, such as peripheral blood mononucleated cells (PBMCs).

The tumor samples described herein can be obtained directly from a subject, derived from a subject, or derived from samples obtained from a subject, such as cultured cells derived from a biological fluid or tissue sample. The tumor biopsy can be a fresh sample. The fresh sample can be fixed after removal from the subject with any known fixatives (e.g. formalin, Zenker's fixative, or B-5 fixative). The tumor biopsy can also be archived samples, such as frozen samples, cryopreserved samples, of cells obtained directly from a subject or of cells derived from cells obtained from a subject. Preferably, the tumor sample obtained from a subject is a fresh tumor biopsy.

The tumor sample can be obtained from a subject by any means including, but not limited to, tumor biopsy, needle aspirate, scraping, surgical excision, surgical incision, venipuncture, or other means known in the art. A tumor biopsy is a preferred method for obtaining the tumor. The tumor biopsy can be obtained from any cancerous site, for example, a primary tumor or a secondary tumor. A tumor biopsy from a primary tumor is generally preferred. Those skilled in the art will recognize other suitable techniques for obtaining tumor samples.

The tumor sample can be obtained from the subject in a single procedure. The tumor sample can be obtained from the subject repeatedly over a period of time. For example, the tumor sample may be obtained once a day, once a week, monthly, biannually, or annually. Obtaining numerous samples over a period of time can be useful to identify and select new tumor-specific neoantigens. The tumor sample can be obtained from the same tumor or different tumors.

The tumor sample can be obtained from the primary tumor, one or more metastases, and/or individual sites of tumor growth (e.g., bone marrow from different skeletal parts, such as hip, bone, or vertebra). The tumor sample can be obtained from the same site or different site.

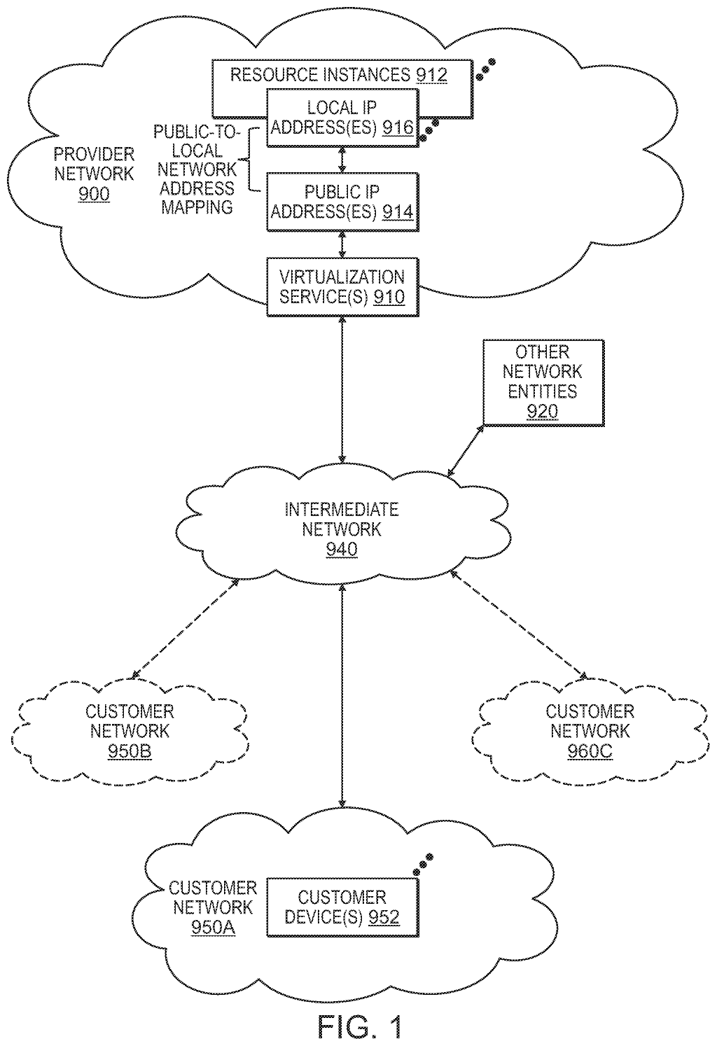

All or any portion of the above described can be implemented on a computing environment such as that illustrated in FIGS. 1-3. FIG. 1 illustrates an example provider network (or “service provider system”) environment according to some embodiments. A provider network 900 may provide resource virtualization to customers via one or more virtualization services 910 that allow customers to purchase, rent, or otherwise obtain instances 912 of virtualized resources, including but not limited to computation and storage resources, implemented on devices within the provider network or networks in one or more data centers. Local Internet Protocol (IP) addresses 916 may be associated with the resource instances 912; the local IP addresses are the internal network addresses of the resource instances 912 on the provider network 900. In some embodiments, the provider network 900 may also provide public IP addresses 914 and/or public IP address ranges (e.g., Internet Protocol version 4 (IPv4) or Internet Protocol version 6 (IPv6) addresses) that customers may obtain from the provider 900.

Conventionally, the provider network 900, via the virtualization services 910, may allow a customer of the service provider (e.g., a customer that operates one or more client networks 950A-950C including one or more customer device(s) 952) to dynamically associate at least some public IP addresses 914 assigned or allocated to the customer with particular resource instances 912 assigned to the customer. The provider network 900 may also allow the customer to remap a public IP address 914, previously mapped to one virtualized computing resource instance 912 allocated to the customer, to another virtualized computing resource instance 912 that is also allocated to the customer. Using the virtualized computing resource instances 912 and public IP addresses 914 provided by the service provider, a customer of the service provider such as the operator of customer network(s) 950A-950C may, for example, implement customer-specific applications and present the customer's applications on an intermediate network 940, such as the Internet. Other network entities 920 on the intermediate network 940 may then generate traffic to a destination public IP address 914 published by the customer network(s) 950A-950C; the traffic is routed to the service provider data center, and at the data center is routed, via a network substrate, to the local IP address 916 of the virtualized computing resource instance 912 currently mapped to the destination public IP address 914. Similarly, response traffic from the virtualized computing resource instance 912 may be routed via the network substrate back onto the intermediate network 940 to the source entity 920.

Local IP addresses, as used herein, refer to the internal or “private” network addresses, for example, of resource instances in a provider network. Local IP addresses can be within address blocks reserved by Internet Engineering Task Force (IETF) Request for Comments (RFC) 1918 and/or of an address format specified by IETF RFC 4193 and may be mutable within the provider network. Network traffic originating outside the provider network is not directly routed to local IP addresses; instead, the traffic uses public IP addresses that are mapped to the local IP addresses of the resource instances. The provider network may include networking devices or appliances that provide network address translation (NAT) or similar functionality to perform the mapping from public IP addresses to local IP addresses and vice versa.

Public IP addresses are Internet mutable network addresses that are assigned to resource instances, either by the service provider or by the customer. Traffic routed to a public IP address is translated, for example via 1:1 NAT, and forwarded to the respective local IP address of a resource instance.

Some public IP addresses may be assigned by the provider network infrastructure to particular resource instances; these public IP addresses may be referred to as standard public IP addresses, or simply standard IP addresses. In some embodiments, the mapping of a standard IP address to a local IP address of a resource instance is the default launch configuration for all resource instance types.

At least some public IP addresses may be allocated to or obtained by customers of the provider network 900; a customer may then assign their allocated public IP addresses to particular resource instances allocated to the customer. These public IP addresses may be referred to as customer public IP addresses, or simply customer IP addresses. Instead of being assigned by the provider network 900 to resource instances as in the case of standard IP addresses, customer IP addresses may be assigned to resource instances by the customers, for example via an API provided by the service provider. Unlike standard IP addresses, customer IP addresses are allocated to customer accounts and can be remapped to other resource instances by the respective customers as necessary or desired. A customer IP address is associated with a customer's account, not a particular resource instance, and the customer controls that IP address until the customer chooses to release it. Unlike conventional static IP addresses, customer IP addresses allow the customer to mask resource instance or availability zone failures by remapping the customer's public IP addresses to any resource instance associated with the customer's account. The customer IP addresses, for example, enable a customer to engineer around problems with the customer's resource instances or software by remapping customer IP addresses to replacement resource instances.

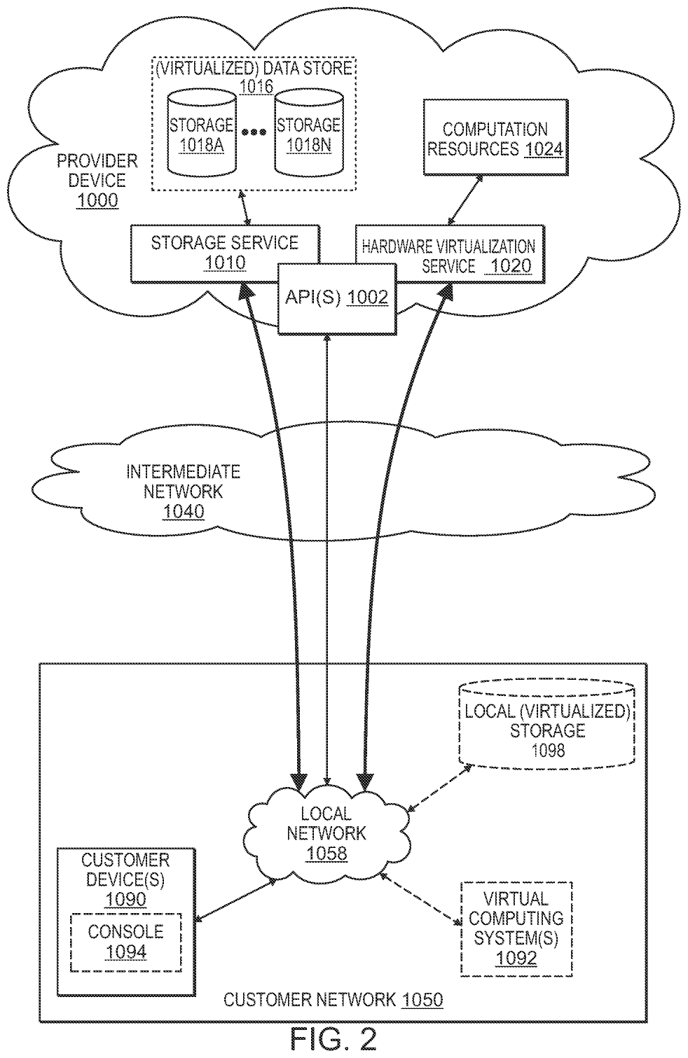

FIG. 2 is a block diagram of an example provider network that provides a storage service and a hardware virtualization service to customers, according to some embodiments. Hardware virtualization service 1020 provides multiple computation resources 1024 (e.g., VMs) to customers. The computation resources 1024 may, for example, be rented or leased to customers of the provider network 1000 (e.g., to a customer that implements customer network 1050). Each computation resource 1024 may be provided with one or more local IP addresses. Provider network 1000 may be configured to route packets from the local IP addresses of the computation resources 1024 to public Internet destinations, and from public Internet sources to the local IP addresses of computation resources 1024.

Provider network 1000 may provide a customer network 1050, for example coupled to intermediate network 1040 via local network 1056, the ability to implement virtual computing systems 1092 via hardware virtualization service 1020 coupled to intermediate network 1040 and to provider network 1000. In some embodiments, hardware virtualization service 1020 may provide one or more APIs 1002, for example a web services interface, via which a customer network 1050 may access functionality provided by the hardware virtualization service 1020, for example via a console 1094 (e.g., a web-based application, standalone application, mobile application, etc.). In some embodiments, at the provider network 1000, each virtual computing system 1092 at customer network 1050 may correspond to a computation resource 1024 that is leased, rented, or otherwise provided to customer network 1050.

From an instance of a virtual computing system 1092 and/or another customer device 1090 (e.g., via console 1094), the customer may access the functionality of storage service 1010, for example via one or more APIs 1002, to access data from and store data to storage resources 1018A-1018N of a virtual data store 1016 (e.g., a folder or “bucket”, a virtualized volume, a database, etc.) provided by the provider network 1000. In some embodiments, a virtualized data store gateway (not shown) may be provided at the customer network 1050 that may locally cache at least some data, for example frequently-accessed or critical data, and that may communicate with storage service 1010 via one or more communications channels to upload new or modified data from a local cache so that the primary store of data (virtualized data store 1016) is maintained. In some embodiments, a user, via a virtual computing system 1092 and/or on another customer device 1090, may mount and access virtual data store 1016 volumes via storage service 1010 acting as a storage virtualization service, and these volumes may appear to the user as local (virtualized) storage 1098.

While not shown in FIG. 2, the virtualization service(s) may also be accessed from resource instances within the provider network 1000 via API(s) 1002. For example, a customer, appliance service provider, or other entity may access a virtualization service from within a respective virtual network on the provider network 1000 via an API 1002 to request allocation of one or more resource instances within the virtual network or within another virtual network.

### Illustrative Systems

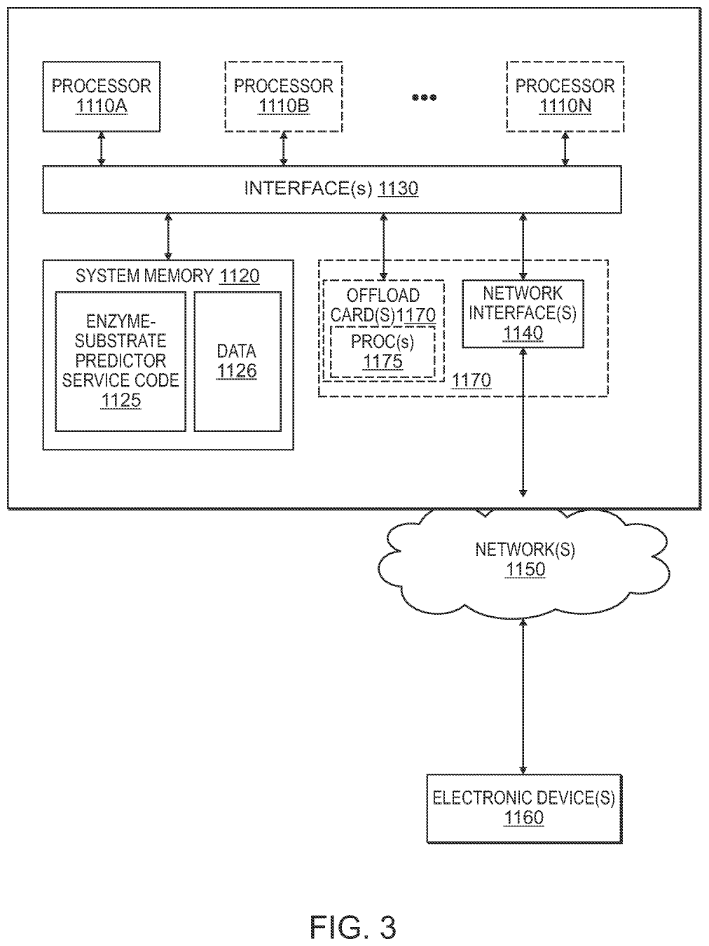

In some embodiments, a system that implements a portion or all of the techniques described herein may include a general-purpose computer system that includes or is configured to access one or more computer-accessible media, such as computer system 1100 illustrated in FIG. 3. In the illustrated embodiment, computer system 1100 includes one or more processors 1110 coupled to a system memory 1120 via an input/output (I/O) interface 1130. Computer system 1100 further includes a network interface 1140 coupled to I/O interface 1130. While FIG. 3 shows computer system 1100 as a single computing device, in various embodiments a computer system 1100 may include one computing device or any number of computing devices configured to work together as a single computer system 1100.

In various embodiments, computer system 1100 may be a uniprocessor system including one processor 1110, or a multiprocessor system including several processors 1110 (e.g., two, four, eight, or another suitable number). Processors 1110 may be any suitable processors capable of executing instructions. For example, in various embodiments, processors 1110 may be general-purpose or embedded processors implementing any of a variety of instruction set architectures (ISAs), such as the x86, ARM, PowerPC, SPARC, or MIPS ISAs, or any other suitable ISA. In multiprocessor systems, each of processors 1110 may commonly, but not necessarily, implement the same ISA.

System memory 1120 may store instructions and data accessible by processor(s) 1110. In various embodiments, system memory 1120 may be implemented using any suitable memory technology, such as random-access memory (RAM), static RAM (SRAM), synchronous dynamic RAM (SDRAM), nonvolatile/Flash-type memory, or any other type of memory. In the illustrated embodiment, program instructions and data implementing one or more desired functions, such as those methods, techniques, and data described above are shown stored within system memory 1120 as enzyme-substrate predictor service code 1125 and data 1126.

In one embodiment, I/O interface 1130 may be configured to coordinate I/O traffic between processor 1110, system memory 1120, and any peripheral devices in the device, including network interface 1140 or other peripheral interfaces. In some embodiments, I/O interface 1130 may perform any necessary protocol, timing or other data transformations to convert data signals from one component (e.g., system memory 1120) into a format suitable for use by another component (e.g., processor 1110). In some embodiments, I/O interface 1130 may include support for devices attached through various types of peripheral buses, such as a variant of the Peripheral Component Interconnect (PCI) bus standard or the Universal Serial Bus (USB) standard, for example. In some embodiments, the function of I/O interface 1130 may be split into two or more separate components, such as a north bridge and a south bridge, for example. Also, in some embodiments some or all of the functionality of I/O interface 1130, such as an interface to system memory 1120, may be incorporated directly into processor 1110.

Network interface 1140 may be configured to allow data to be exchanged between computer system 1100 and other devices 1160 attached to a network or networks 1150. In various embodiments, network interface 1140 may support communication via any suitable wired or wireless general data networks, such as types of Ethernet network, for example. Additionally, network interface 1140 may support communication via telecommunications/telephony networks such as analog voice networks or digital fiber communications networks, via storage area networks (SANs) such as Fibre Channel SANs, or via I/O any other suitable type of network and/or protocol.

In some embodiments, a computer system 1100 includes one or more offload cards 1170 (including one or more processors 1175, and possibly including the one or more network interfaces 1140) that are connected using an I/O interface 1130 (e.g., a bus implementing a version of the Peripheral Component Interconnect Express (PCI-E) standard, or another interconnect such as a QuickPath interconnect (QPI) or UltraPath interconnect (UPI)). For example, in some embodiments the computer system 1100 may act as a host electronic device (e.g., operating as part of a hardware virtualization service) that hosts compute instances, and the one or more offload cards 1170 execute a virtualization manager that can manage compute instances that execute on the host electronic device. As an example, in some embodiments the offload card(s) 1170 can perform compute instance management operations such as pausing and/or un-pausing compute instances, launching and/or terminating compute instances, performing memory transfer/copying operations, etc. These management operations may, in some embodiments, be performed by the offload card(s) 1170 in coordination with a hypervisor (e.g., upon a request from a hypervisor) that is executed by the other processors 1110A-1110N of the computer system 1100. However, in some embodiments the virtualization manager implemented by the offload card(s) 1170 can accommodate requests from other entities (e.g., from compute instances themselves), and may not coordinate with (or service) any separate hypervisor.

In some embodiments, system memory 1120 may be one embodiment of a computer-accessible medium configured to store program instructions and data as described above. However, in other embodiments, program instructions and/or data may be received, sent, or stored upon different types of computer-accessible media. Generally speaking, a computer-accessible medium may include non-transitory storage media or memory media such as magnetic or optical media, e.g., disk or DVD/CD coupled to computer system 1100 via I/O interface 1130. A non-transitory computer-accessible storage medium may also include any volatile or non-volatile media such as RAM (e.g., SDRAM, double data rate (DDR) SDRAM, SRAM, etc.), read only memory (ROM), etc., that may be included in some embodiments of computer system 1100 as system memory 1120 or another type of memory. Further, a computer-accessible medium may include transmission media or signals such as electrical, electromagnetic, or digital signals, conveyed via a communication medium such as a network and/or a wireless link, such as may be implemented via network interface 1140.

Various embodiments discussed or suggested herein can be implemented in a wide variety of operating environments, which in some cases can include one or more user computers, computing devices, or processing devices which can be used to operate any of a number of applications. User or client devices can include any of a number of general-purpose personal computers, such as desktop or laptop computers running a standard operating system, as well as cellular, wireless, and handheld devices running mobile software and capable of supporting a number of networking and messaging protocols. Such a system also can include a number of workstations running any of a variety of commercially available operating systems and other known applications for purposes such as development and database management. These devices also can include other electronic devices, such as dummy terminals, thin-clients, gaming systems, and/or other devices capable of communicating via a network.

Most embodiments utilize at least one network that would be familiar to those skilled in the art for supporting communications using any of a variety of widely-available protocols, such as Transmission Control Protocol/Internet Protocol (TCP/IP), File Transfer Protocol (FTP), Universal Plug and Play (UPnP), Network File System (NFS), Common Internet File System (CIFS), Extensible Messaging and Presence Protocol (XMPP), AppleTalk, etc. The network(s) can include, for example, a local area network (LAN), a wide-area network (WAN), a virtual private network (VPN), the Internet, an intranet, an extranet, a public switched telephone network (PSTN), an infrared network, a wireless network, and any combination thereof.

In embodiments utilizing a web server, the web server can run any of a variety of server or mid-tier applications, including HTTP servers, File Transfer Protocol (FTP) servers, Common Gateway Interface (CGI) servers, data servers, Java servers, business application servers, etc. The server(s) also may be capable of executing programs or scripts in response requests from user devices, such as by executing one or more Web applications that may be implemented as one or more scripts or programs written in any programming language, such as Java®, C, C# or C++, or any scripting language, such as Perl, Python, PUP, or TCL, as well as combinations thereof. The server(s) may also include database servers, including without limitation those commercially available from Oracle®, Microsoft®, Sybase®, IBM®, etc. The database servers may be relational or non-relational (e.g., “NoSQL”), distributed or non-distributed, etc.

Environments disclosed herein can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers, or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch screen, or keypad), and/or at least one output device (e.g., a display device, printer, or speaker). Such a system may also include one or more storage devices, such as disk drives, optical storage devices, and solid-state storage devices such as random-access memory (RAM) or read-only memory (ROM), as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or web browser. It should be appreciated that alternate embodiments may have numerous variations from that described above. For example, customized hardware might also be used and/or particular elements might be implemented in hardware, software (including portable software, such as applets), or both. Further, connection to other computing devices such as network input/output devices may be employed.

Storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, Electrically Erasable Programmable Read-Only Memory (EEPROM), flash memory or other memory technology, Compact Disc-Read Only Memory (CD-ROM), Digital Versatile Disk (DVD) or other optical storage, magnetic cassettes, magnetic tape, magnetic disk storage or other magnetic storage devices, or any other medium which can be used to store the desired information and which can be accessed by a system device. Based on the disclosure and teachings provided herein, a person of ordinary skill in the art will appreciate other ways and/or methods to implement the various embodiments.

In the preceding description, various embodiments are described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Bracketed text and blocks with dashed borders (e.g., large dashes, small dashes, dot-dash, and dots) are used herein to illustrate optional operations that add additional features to some embodiments. However, such notation should not be taken to mean that these are the only options or optional operations, and/or that blocks with solid borders are not optional in certain embodiments.

Reference numerals with suffix letters may be used to indicate that there can be one or multiple instances of the referenced entity in various embodiments, and when there are multiple instances, each does not need to be identical but may instead share some general traits or act in common ways. Further, the particular suffixes used are not meant to imply that a particular amount of the entity exists unless specifically indicated to the contrary. Thus, two entities using the same or different suffix letters may or may not have the same number of instances in various embodiments.

References to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Moreover, in the various embodiments described above, unless specifically noted otherwise, disjunctive language such as the phrase “at least one of A, B, or C” is intended to be understood to mean either A, B, or C, or any combination thereof (e.g., A, B, and/or C). As such, disjunctive language is not intended to, nor should it be understood to, imply that a given embodiment requires at least one of A, at least one of B, or at least one of C to each be present.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

### EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

### EXEMPLIFICATION

The following examples are provided for illustrative purposes only, and are not intended to be limiting in any way.

### Example 1

| Variant | chr2 g.122519017G > A |
|---|---|
| IGV locus | chr2: 122519017 |
| Gene name | TSN |
| Gene ID | ENSG00000211460 |
| RNA reads supporting variant allele | 32 |
| RNA reads supporting reference allele | 71 |
| RNA reads supporting other alleles | 0 |
| RNA TPM | 0 |
| Cluster ID | 15 |
| Cluster Assignment Probability | 0.114 |
| Cellular Prevalence | 0.99 |

Predicted Effect

| | |
|---|---|
| Effect type | Substitution |
| Transcript name | TSN-001 |
| Transcript ID | ENST00000389682 |
| Effect description | p.R97H |

MHC Class I Vaccine Peptide Candidate

(SEQ ID NO: 26)
FVLQ [symbol] LVFL

| | |
|---|---|
| Length | 9 |
| MHC Class I immunogenicity score | 0.002 |
| MHC Class I immunogenicity-binding score | 0.001 |
| MHC Class I unscaled immunogenicity score | 0.165 |
| MHC Class I unscaled immunogenicity-binding score | 0.134 |
| MHC Class I binding score | 0.998 |
| RNA TPM | 0 |
| Max coding sequence coverage | 30 |
| Mutant amino acids | 1 |
| Mutation distance from edge | 4 |

Predicted Mutant Epitopes

| | | | | | | | | | | WT | WT | | |
|---|---|---|---|---|---|---|---|---|---|---|---|---|---|
| | | | | Immuno- | Presen- | | | | | immuno- | presen- | | WT |
| | | | Dis- | genicity | tation | | Binding | | | genicity | tation | WT | binding |
| | | SEQ | tance | prob- | prob- | Binding | prob- | | SEQ | prob- | prob- | binding | prob- |
| MHC | | ID | to | ability | ability | affinity | ability | WT | ID | ability | ability | affinity | ability |
| allele | Sequence | NO: | self | (%) | (%) | (nM) | (%) | sequence | NO: | (%) | (%) | (nM) | (nM) |
| A*02: | FVLQHLVF | 26 | 5 | 48.82 | 99.22 | 10.92 | 97.5 | FVLQRLVF | 39 | 39.43 | 96.2 | 61.54 | 89.34 |
| 01 | L | | | | | | | L | | | | | |
| C*03: | FVLQHLVF | 26 | 5 | 50.28 | 99.64 | 187.65 | 75.67 | FVLQRLVF | 39 | 44.91 | 98.03 | 557.72 | 54.14 |
| 04 | L | | | | | | | L | | | | | |
| 8*58: | FVLQHLVF | 26 | 5 | 0.49 | 17.65 | 3447.14 | 18.95 | FVLQRLVF | 39 | 0.49 | 20.95 | 8040.31 | 9.92 |
| 01 | L | | | | | | | L | | | | | |
| C*05: | FVLQHLVF | 26 | 5 | 16.27 | 84.55 | 772.04 | 46.93 | FVLQRLVF | 39 | 5.34 | 71.45 | 1137.74 | 38.52 |
| 01 | L | | | | | | | L | | | | | |
| A*03: | FVLQHLVF | 26 | 5 | 0.49 | 21.62 | 15646.88 | 5.74 | FVLQRLVF | 39 | 0.5 | 29.02 | 17796.40 | 5.15 |
| 01 | L | | | | | | | L | | | | | |
| B*40: | FVLQHLVF | 26 | 5 | 0.49 | 1.68 | 31478.40 | 3.17 | FVLQRLVF | 39 | 0.49 | 1.14 | 39538.96 | 2.60 |
| 01 | L | | | | | | | L | | | | | |

Example 1 illustrates a short MHC Class I vaccine peptide candidate and predicted mutant epitopes for an example variant, according to an example embodiment. In this example, the boxed letter “H” represents a mutated subsequence of the vaccine peptide sequence “FVLQHLVFL”. According to one or more of the methods described elsewhere herein, one or more mutant epitopes may be predicted, and an immunogenicity score may be generated. According to this example, the immunogenicity score may indicate a probability that at least one epitope in a longer peptide sequence is immunogenic on at least one of the subject's MHC Class I alleles. The MHC Class I binding score may indicate a probability that the peptide binds to at least one of the subject's MHC Class I alleles. Additionally, the length may indicate a number of amino acids in the sequence, which may be used to distinguish between short and long neoantigens. The MHC Class I immunogenicity-binding score may be determined by multiplying the MHC Class I immunogenicity score by the MHC Class I binding score. RNA TPM may indicate a number of RNA reads normalized per gene length and sequencing depth, in transcripts per million (TPM). Max coding sequence coverage may indicate a number of RNA reads covering the vaccine peptide sequence.

### Example 2

| Variant | chr2 g.183622543A > G |
|---|---|
| IGV locus | chr2: 183622543 |
| Gene name | DNAJC10 |
| Gene ID | ENSG00000077232 |
| RNA reads supporting variant allele | 158 |
| RNA reads supporting reference allele | 221 |
| RNA reads supporting other alleles | 0 |
| RNA TPM | 0 |
| Cluster ID | 12 |
| Cluster Assignment Probability | 0.203 |
| Cellular Prevalence | 1.0 |

Predicted Effect

| | |
|---|---|
| Effect type | Substitution |
| Transcript name | DNAJC10-001 |
| Transcript ID | ENST00000264065 |
| Effect description | p.Y645C |

MHC Class I Vaccine Peptide Candidate

(SEQ ID NO: 7)
KA [symbol] HYHSYNGW

| | |
|---|---|
| Length | 11 |
| MHC Class I immunogenicity score | 0.003 |
| MHC Class I immunogenicity-binding score | 0.001 |
| MHC Class I unscaled immunogenicity score | 0.297 |
| MHC Class I unscaled immunogenicity-binding score | 0.12 |
| MHC Class I binding score | 0.518 |
| RNA TPM | 0 |
| Max coding sequence coverage | 155 |
| Mutant amino acids | 1 |
| Mutation distance from edge | 2 |

Predicted Mutant Epitopes

| | | | | | | | | | | WT | WT | | |
|---|---|---|---|---|---|---|---|---|---|---|---|---|---|
| | | | | Immuno- | Presen- | | | | | immuno- | presen- | | WT |
| | | | Dis- | genicity | tation | | Binding | | | genicity | tation | WT | binding |
| | | SEQ | tance | prob- | prob- | Binding | prob- | | SEQ | prob- | prob- | binding | prob- |
| MHC | | ID | to | ability | ability | affinity | ability | WT | ID | ability | ability | affinity | ability |
| allele | Sequence | NO: | self | (%) | (%) | (nM) | (%) | sequence | NO: | (%) | (%) | (nM) | (nM) |
| A*02: | KACHYHSYN | 7 | 9 | 0.49 | 4.26 | 44071.60 | 2.37 | KAYHYHSYN | 16 | 0.49 | 5.76 | 40795.97 | 2.53 |
| 01 | GW | | | | | | | GW | | | | | |
| C*03: | KACHYHSYN | 7 | 9 | 0.49 | 1.42 | 21711.91 | 4.36 | KAYHYHSYN | 16 | 0.49 | 9.52 | 11167.03 | 7.60 |
| 04 | GW | | | | | | | GW | | | | | |
| B*58: | KACHYHSYN | 7 | 9 | 51.49 | 99.98 | 978.41 | 41.74 | KAYHYHSYN | 16 | 51.45 | 99.97 | 861.39 | 44.51 |
| 01 | GW | | | | | | | GW | | | | | |
| C*05: | KACHYHSYN | 7 | 9 | 0.49 | 6.4 | 11904.31 | 7.21 | KAYHYHSYN | 16 | 0.49 | 11.4 | 10147.49 | 8.22 |
| 01 | GW | | | | | | | GW | | | | | |
| A*03: | KACHYHSYN | 7 | 9 | 0.49 | 1.09 | 44999.90 | 2.33 | KAYHYHSYN | 16 | 0.49 | 3.59 | 40703.25 | 2.54 |
| 01 | GW | | | | | | | GW | | | | | |
| B*40: | KACHYHSYN | 7 | 9 | 0.49 | 2.11 | 45584.43 | 2.3 | KAYHYHSYN | 16 | 0.49 | 2.3 | 39413.24 | 2.61 |
| 01 | GW | | | | | | | GW | | | | | |

Example 2 illustrates another short MHC Class I vaccine peptide candidate and predicted mutant epitopes for an example variant, according to an example embodiment. In this example, the box around letter “C” represents a mutated subsequence of the vaccine peptide sequence “KACHYHSYNGW”.

### Example 3

| Variant | chr17 g.17380542T > C |
|---|---|
| IGV locus | chr17: 17380542 |
| Gene name | MED9 |
| Gene ID | ENSG00000141026 |
| RNA reads supporting variant allele | 20 |
| RNA reads supporting reference allele | 0 |
| RNA reads supporting other alleles | 0 |
| RNA TPM | 0 |
| Cluster ID | 15 |
| Cluster Assignment Probability | 0.113 |
| Cellular Prevalence | 0.99 |

Predicted Effect

| | |
|---|---|
| Effect type | Substitution |
| Transcript name | MED9-001 |
| Transcript ID | ENST00000268711 |
| Effect description | p.Y63H |

MHC Class I Vaccine Peptide Candidate

REEEN [symbol] SFL (SEQ ID NO: 50)

| | |
|---|---|
| Length | 9 |
| MHC Class I immunogenicity | 0.001 |
| score | |
| MHC Class 1 immunogenicity- | 0.001 |
| binding score | |
| MHC Class I unsealed | 0.081 |
| immunogenicity score | |
| MHC Class I unsealed | 0.077 |
| immunogenicity-binding | |
| score | |
| MHC Class I binding score | 0.995 |
| RNATPM | 0 |
| Max coding sequence | 19 |
| coverage | |
| Mutant amino acids | 1 |
| Mutation distance from edge | 3 |

| | | | | | | | | | | WT | | | |
|---|---|---|---|---|---|---|---|---|---|---|---|---|---|
| | | | | Immuno- | Presen- | | | | | immuno- | WT | | WT |
| | | | Dis- | genicity | tation | | Binding | | | genicity | presen- | WT | binding |
| | | SEQ | tance | prob- | prob- | Binding | prob- | | SEQ | prob- | tation | binding | prob- |
| MHC | | ID | to | ability | ability | affinity | ability | WT | ID | ability | prob- | affinity | ability |
| allele | Sequence | NO: | self | (%) | (%) | (nM) | (%) | sequence | NO: | (%) | ability | (nM) | (nM) |
| A*02: | REEENHSF | 50 | 5 | 0.66 | 44.12 | 5351.98 | 13.66 | REEENYSF | 59 | 0.56 | 38.94% | 4222.01 | 16.34 |
| 01 | L | | | | | | | L | | | | | |
| C*03: | REEENHSF | 50 | 5 | 0.49 | 1.01 | 15388.33 | 5.82 | REEENYSF | 59 | 0.49 | 0.8% | 12963.82 | 6.72 |
| 04 | L | | | | | | | L | | | | | |
| 8*58: | REEENHSF | 50 | 5 | 0.49 | 9.37 | 33411.26 | 3.01 | REEENYSF | 59 | 0.49 | 9.75% | 29984.21 | 3.31 |
| 01 | L | | | | | | | L | | | | | |
| C*05: | REEENHSF | 50 | 5 | 0.5 | 30.3 | 757.61 | 47.35 | REEENYSF | 59 | 0.5 | 29.07% | 1401.40 | 34.23 |
| 01 | L | | | | | | | L | | | | | |
| A*03: | REEENHSF | 50 | 5 | 0.49 | 3.01 | 44138.81 | 2.37 | REEENYSF | 59 | 0.49 | 3.81% | 43067.19 | 2.42 |
| 01 | L | | | | | | | L | | | | | |
| B*40: | REEENHSF | 50 | 5 | 51.54 | 99.99 | 4.38 | 98.87 | REEENYSF | 59 | 51.49 | 99.98% | 3.61 | 99.05 |
| 01 | L | | | | | | | L | | | | | |

Example 3 illustrates another short MHC Class I vaccine peptide candidate and predicted mutant epitopes for an example variant. In this example, the box around letter “H” represents a mutated subsequence of the vaccine peptide sequence “REEENHISFL”.

### Example 4

| Variant | chr2 g.122519017G > A |
|---|---|
| IGV locus | chr2: 122519017 |
| Gene name | TSN |
| Gene ID | ENSG00000211460 |
| RNA reads supporting variant allele | 32 |
| RNA reads supporting reference allele | 71 |
| RNA reads supporting other alleles | 0 |
| RNA TPM | 0 |
| Cluster ID | 15 |
| Cluster Assignment Probability | 0.114 |
| Cellular Prevalence | 0.99 |

Predicted Effect

| | |
|---|---|
| Effect type | Substitution |
| Transcript name | TSN-001 |
| Transcript ID | ENST00000389682 |
| Effect description | p.R97H |

MHC Class I Vaccine Peptide

(SEQ ID NO: 21)
HEHWRFVLQ [symbol] LVFLAAFVV

| | |
|---|---|
| Length | 19 |
| MHC Class I immunogenicity score | 0.003 |
| MHC Class I immunogenicity-binding score | 0.002 |
| MHC Class I unscaled immunogenicity score | 0.289 |
| MHC Class I unscaled immunogenicity-binding score | 0.242 |
| MHC Class I binding score | 1.0 |
| RNA TPM | 0 |
| Max coding sequence coverage | 30 |
| Mutant amino acids | 1 |
| Mutation distance from edge | 9 |

Predicted Mutant Epitopes

| | | | | | | | | | | WT | WT | | |
|---|---|---|---|---|---|---|---|---|---|---|---|---|---|
| | | | | Immuno- | Presen- | | | | | immuno- | presen- | | WT |
| | | | Dis- | genicity | tation | | Binding | | | genicity | tation | WT | binding |
| | | SEQ | tance | prob- | prob- | Binding | prob- | | SEQ | prob- | prob- | binding | prob- |
| MHC | | ID | to | ability | ability | affinity | ability | WT | ID | ability | ability | affinity | ability |
| allele | Sequence | NO: | self | (%) | (%) | (nM) | (%) | sequence | NO: | (%) | (%) | (nM) | (nM) |
| A*02: | WRFVLQHL | 22 | 5 | 0.49 | 19.9 | 8291.11 | 9.68 | WRFVLQRLV | 35 | 0.49 | 10.81 | 16664.99 | 5.45 |
| 01 | V | | | | | | | | | | | | |
| A*02: | VLQHLVFL | 23 | 5 | 46.92 | 98.65 | 37.26 | 92.9 | VLQRLVFLA | 36 | 37.72 | 95.58 | 80.37 | 86.86 |
| 01 | A | | | | | | | | | | | | |
| A*02: | EHWRFVLQ | 24 | 5 | 0.49 | 10.55 | 23862.32 | 4.02 | EHWRFVLQRL | 37 | 0.49 | 11.23 | 23713.47 | 4.04 |
| 01 | HL | | | | | | | | | | | | |
| A*02: | HLVFLAAF | 25 | 5 | 46.05 | 98.38 | 56.76 | 90.0 | RLVFLAAFV | 38 | 43.24 | 97.49 | 34.11 | 93.40 |
| 01 | V | | | | | | | | | | | | |
| A*02: | FVLQHLVF | 26 | 5 | 48.82 | 99.22 | 10.92 | 97.5 | FVLQRLVFL | 39 | 39.43 | 96.2 | 61.54 | 89.34 |
| 01 | L | | | | | | | | | | | | |
| C*03: | WRFVLQHL | 27 | 5 | 0.49 | 8.75 | 13370.73 | 6.55 | WRFVLQRL | 40 | 0.49 | 5.54 | 15406.63 | 5.82 |
| 04 | | | | | | | | | | | | | |
| C*03: | EHWRFVLQ | 28 | 5 | 0.49 | 4.07 | 25352.75 | 3.82 | EHWRFVLQR | 41 | 0.49 | 3.77 | 25601.34 | 3.78 |
| 04 | H | | | | | | | | | | | | |
| C*03: | HLVFLAAF | 29 | 5 | 0.49 | 26.59 | 24998.81 | 3.86 | RLVFLAAFVV | 42 | 0.49 | 17.09 | 28299.16 | 3.47 |
| 04 | VV | | | | | | | | | | | | |
| C*03: | LQHLVFLA | 30 | 5 | 0.5 | 28.99 | 5481.79 | 13.41 | LQRLVFLAA | 43 | 0.49 | 20.57 | 5669.23 | 13.06 |
| 04 | A | | | | | | | | | | | | |
| C*03: | FVLQHLVF | 26 | 5 | 50.28 | 99.64 | 187.65 | 75.67 | FVLQRLVFL | 39 | 44.91 | 98.03 | 557.72 | 54.14 |
| 04 | L | | | | | | | | | | | | |
| 8*58: | HLVFLAAF | 29 | 5 | 0.49 | 26.41 | 19190.01 | 4.84 | RLVFLAAFVV | 42 | 0.49 | 22.32 | 16047.60 | 5.62 |
| 01 | VV | | | | | | | | | | | | |
| B*58: | RFVLQHLV | 31 | 5 | 2.52 | 63.07 | 5277.70 | 13.8 | RFVLQRLVF | 44 | 1.04 | 52.37 | 7305.64 | 10.71 |
| 01 | F | | | | | | | | | | | | |
| B*58: | EHWRFVLQ | 24 | 5 | 0.49 | 10.6 | 39268.57 | 2.62 | EHWRFVLQRL | 37 | 0.49 | 8.64 | 43201.16 | 2.41 |
| 01 | HL | | | | | | | | | | | | |
| C*05: | HWRFVLQH | 32 | 5 | 0.49 | 14.12 | 5641.02 | 13.11 | HWRFVLQRL | 45 | 0.49 | 11.01 | 8206.61 | 9.76 |
| 01 | L | | | | | | | | | | | | |
| C*05: | HLVFLAAF | 29 | 5 | 1.43 | 56.51 | 1733.52 | 30.11 | RLVFLAAFVV | 42 | 1.32 | 55.54 | 2832.31 | 21.78 |
| 01 | VV | | | | | | | | | | | | |
| C*05: | FVLQHLVF | 26 | 5 | 16.27 | 84.55 | 772.04 | 46.93 | FVLQRLVFL | 39 | 5.34 | 71.45 | 1137.74 | 38.52 |
| 01 | L | | | | | | | | | | | | |
| A*03: | WRFVLQHL | 27 | 5 | 0.49 | 7.29 | 43942.78 | 2.38 | WRFVLQRL | 40 | 0.49 | 8.46 | 42281.07 | 2.46 |
| 01 | | | | | | | | | | | | | |
| A*03: | HLVFLAAF | 29 | 5 | 0.5 | 30.35 | 20169.84 | 4.64 | RLVFLAAFVV | 42 | 0.81 | 48.5 | 10093.13 | 8.26 |
| 01 | VV | | | | | | | | | | | | |
| A*03: | FVLQHLVF | 26 | 5 | 0.49 | 21.62 | 15646.88 | 5.74 | FVLQRLVFL | 39 | 0.5 | 29.02 | 17796.40 | 5.15 |
| 01 | L | | | | | | | | | | | | |
| B*40: | VLQHLVFL | 33 | 5 | 0.49 | 9.91 | 44833.00 | 2.33 | VLQRLVFL | 46 | 0.49 | 8.99 | 46142.77 | 2.28 |
| 01 | | | | | | | | | | | | | |
| B*40: | HLVFLAAF | 29 | 5 | 0.49 | 4.03 | 36317.27 | 2.8 | RLVFLAAFVV | 42 | 0.49 | 2.8 | 34051.19 | 2.96 |
| 01 | VV | | | | | | | | | | | | |
| B*40: | HEHWRFVL | 34 | 5 | 1.71 | 58.63 | 4861.01 | 14.7 | HEHWRFVLQR | 47 | 0.52 | 33.92 | 8264.02 | 9.71 |
| 01 | QHL | | | | | | | L | | | | | |

### Example 5

MHC Class II Vaccine Peptide

(SEQ ID NO: 21)
HEHWRFVLQ [symbol] LVFLAAFVV

| Transcript name | TSN-001 |
|---|---|
| Length | 19 |
| MHC Class I immunogenicity score | 0.003 |
| MHC Class II immunogenicity score | 0.72 |
| RNA TPM | 0 |
| Max coding sequence coverage | 30 |
| Mutant amino acids | 1 |
| Mutation distance from edge | 9 |

### Predicted Mutant Epitopes

| | | | | | | | WT | WT |
|---|---|---|---|---|---|---|---|---|
| | | SEQ | Binding | Binding | | SEQ | binding | binding |
| MHC | | ID | affinity | probability | | ID | affinity | probability |
| allele | Sequence | NO: | (nM) | (%) | WT sequence | NO: | (nM) | (nM) |
| DQB105:01 | HEHWRFVLQHLVFLAAFVV | 21 | 2422.37 | 11.88 | HEHWRFVLQRLVFLAAFVV | 48 | 2739.74 | 11.41 |
| DQB103:02 | HEHWRFVLQHLVFLAAFVV | 21 | 5521.58 | 9.02 | HEHWRFVLQRLVFLAAFVV | 48 | 3673.07 | 10.35 |
| DRB101:03 | HEHWRFVLQHLVFLAAFVV | 21 | 7621.53 | 8.08 | HEHWRFVLQRLVFLAAFVV | 48 | 10911.09 | 7.14 |
| DPA101:03 | HEHWRFVLQHLVFLAAFVV | 21 | 4225.83 | 9.87 | HEHWRFVLQRLVFLAAFVV | 48 | 7644.05 | 8.07 |
| DQA103:01 | HEHWRFVLQHLVFLAAFVV | 21 | 2331.70 | 12.03 | HEHWRFVLQRLVFLAAFVV | 48 | 3389.87 | 10.63 |
| DQA101:01 | HEHWRFVLQHLVFLAAFVV | 21 | 108.32 | 30.1 | HEHWRFVLQRLVFLAAFVV | 48 | 141.65 | 28.03 |
| DRB104:01 | HEHWRFVLQHLVFLAAFVV | 21 | 2593.30 | 11.62 | HEHWRFVLQRLVFLAAFVV | 48 | 2613.22 | 11.59 |
| DRB401:03 | HEHWRFVLQHLVFLAAFVV | 21 | 729.66 | 17.43 | HEHWRFVLQRLVFLAAFVV | 48 | 1063.17 | 15.50 |
| DPB102:01 | HEHWRFVLQHLVFLAAFVV | 21 | 344.21 | 21.85 | HEHWRFVLQRLVFLAAFVV | 48 | 541.72 | 19.09 |
| DPB103:01 | HEHWRFVLQHLVFLAAFVV | 21 | 5709.67 | 8.92 | HEHWRFVLQRLVFLAAFVV | 48 | 5560.11 | 9.00 |

In Examples 4 and 5 above, short sequence “FVLQHLVFL” of Example 1 is used to create a sequence for the long MHC Class I vaccine peptide in Example 4 and the long MHC Class II vaccine peptide of Example 5, both including the same short subsequence (e.g., boxed letter “H”) at the center of the sequence. As explained elsewhere herein, amino acids may be added to both sides of the short subsequence, according to the longest neoantigen, such that there is a first maximum number of amino acids flanking each side of the mutated amino acid. Predicted mutant epitopes may be generated or determined for both the MHC Class I vaccine peptide and the MHC Class II vaccine peptide, along with corresponding immunogenicity scores. The MHC Class I immunogenicity score may indicate a probability that at least one epitope in a longer peptide sequence is immunogenic on at least one of the subject's MHC Class I alleles. The MHC Class II immunogenicity score may indicate a probability that at least one epitope in a longer peptide sequence is immunogenic on at least one of the subject's MHC Class II alleles.

### Example 6

| Variant | chr2 g.183622543A > G |
|---|---|
| IGV locus | chr2: 183622543 |
| Gene name | DNAJC10 |
| Gene ID | ENSG00000077232 |
| RNA reads supporting variant allele | 158 |
| RNA reads supporting reference allele | 221 |
| RNA reads supporting other alleles | 0 |
| RNA TPM | 0 |
| Cluster ID | 12 |
| Cluster Assignment Probability | 0.203 |
| Cellular Prevalence | 1.0 |

Predicted Effect

| | |
|---|---|
| Effect type | Substitution |
| Transcript name | DNAJC10-001 |
| Transcript ID | ENST00000264065 |
| Effect description | p.Y645C |

MHC Class I Vaccine Peptide

(SEQ ID NO: 1)
RFFPPKSNKA [symbol] HYHSYNGWNR

| | |
|---|---|
| Length | 21 |
| MHC Class I immunogenicity score | 0.003 |
| MHC Class I immunogenicity-binding score | 0.001 |
| MHC Class I unscaled immunogenicity score | 0.314 |
| MHC Class I unscaled immunogenicity-binding score | 0.121 |
| MHC Class I binding score | 1.0 |
| RNA TPM | 0 |
| Max coding sequence coverage | 155 |
| Mutant amino acids | 1 |
| Mutation distance from edge | 10 |

| | | | | | | | | | | WT | | | |
|---|---|---|---|---|---|---|---|---|---|---|---|---|---|
| | | | | Immuno- | Presen- | | | | | immuno- | | | WT |
| | | | Dis- | genicity | tation | | Binding | | | genicity | | WT | binding |
| | | SEQ | tance | prob- | prob- | Binding | prob- | | SEQ | prob- | WT | binding | prob- |
| MHC | | ID | to | ability | ability | affinity | ability | WT | ID | ability | presen- | affinity | ability |
| allele | Sequence | NO: | self | (%) | (%) | (nM) | (%) | sequence | NO: | (%) | tation | (nM) | (nM) |
| A*02: | FPPKSNKA | 2 | 9 | 0.49 | 17.63 | 27089.93 | 3.61 | FPPKSNKA | 11 | 0.49 | 23.67 | 21218.57 | 4.44 |
| 01 | CHY | | | | | | | YHY | | | | | |
| A*02: | KACHYHS | 3 | 9 | 0.49 | 10.76 | 36125.83 | 2.81 | KAYHYHSY | 12 | 0.49 | 12.37 | 27777.06 | 3.53 |
| 01 | Y | | | | | | | | | | | | |
| A*02: | CHYHSYN | 4 | 9 | 0.49 | 4.43 | 44386.96 | 2.35 | YHYHSYNG | 13 | 0.49 | 9.95 | 40113.94 | 2.57 |
| 01 | G | | | | | | | | | | | | |
| C*03: | KACHYHS | 3 | 9 | 0.49 | 5.82 | 19921.22 | 4.69 | KAYHYHSY | 12 | 0.5 | 28.61 | 5317.78 | 13.72 |
| 04 | Y | | | | | | | | | | | | |
| C*03: | SNKACHY | 5 | 9 | 0.49 | 0.68 | 32847.06 | 3.06 | SNKAYHYH | 14 | 0.49 | 1.22 | 28136.63 | 3.49 |
| 04 | H | | | | | | | | | | | | |
| C*03: | FFPPKSNK | 6 | 9 | 0.49 | 3.28 | 36066.14 | 2.82 | FFPPKSNK | 15 | 0.49 | 3.58 | 33726.03 | 2.99 |
| 04 | A CH | | | | | | | AYH | | | | | |
| B*58: | KACHYHS | 7 | 9 | 51.49 | 99.98 | 978.41 | 41.74 | KAYHYHSY | 16 | 51.45 | 99.97 | 861.39 | 44.51 |
| 01 | YNGW | | | | | | | NGW | | | | | |
| B*58: | RFFPPKSN | 8 | 9 | 0.49 | 7.34 | 30205.95 | 3.28 | RFFPPKSN | 17 | 0.49 | 11.45 | 29175.08 | 3.38 |
| 01 | KAC | | | | | | | KAY | | | | | |
| C*05: | FPPKSNKA | 2 | 9 | 0.49 | 11.28 | 12464.75 | 6.94 | FPPKSNKA | 11 | 0.49 | 14.82 | 9155.75 | 8.94 |
| 01 | CHY | | | | | | | YHY | | | | | |
| C*05: | KACHYHS | 3 | 9 | 0.49 | 10.54 | 9412.87 | 8.74 | KAYHYHSY | 12 | 0.49 | 17.08 | 6445.97 | 11.82 |
| 01 | Y | | | | | | | | | | | | |
| C*05: | FFPPKSNK | 6 | 9 | 0.49 | 12.26 | 14855.78 | 6.0 | FFPPKSNK | 15 | 0.49 | 14.37 | 12684.41 | 6.84 |
| 01 | ACH | | | | | | | AYH | | | | | |
| A*03: | KSNKACH | 9 | 9 | 0.49 | 25.05 | 6670.69 | 11.51 | KSNKAYHY | 18 | 0.49 | 20.09 | 7297.12 | 10.72 |
| 01 | YHSY | | | | | | | HSY | | | | | |
| A*03: | CHYHSYN | 10 | 9 | 0.49 | 1.44 | 25523.83 | 3.79 | YHYHSYNG | 19 | 0.49 | 5.25 | 25878.83 | 3.75 |
| 01 | GWNR | | | | | | | WNR | | | | | |
| B*40: | FPPKSNKA | 2 | 9 | 0.49 | 0.85 | 45076.23 | 2.32 | FPPKSNKA | 11 | 0.49 | 0.67 | 43021.44 | 2.42 |
| 01 | CHY | | | | | | | YHY | | | | | |
| B*40: | KACHYHS | 3 | 9 | 0.49 | 3.22 | 46290.99 | 2.27 | KAYHYHSY | 12 | 0.49 | 1.52 | 42191.59 | 2.46 |
| 01 | Y | | | | | | | | | | | | |
| B*40: | RFFPPKSN | 8 | 9 | 0.49 | 0.86 | 47085.26 | 2.24 | RFFPPKSN | 17 | 0.49 | 0.51 | 34617.29 | 2.92 |
| 01 | KAC | | | | | | | KAY | | | | | |
| B*40: | FFPPKSNK | 6 | 9 | 0.49 | 0.82 | 48492.38 | 2.18 | FFPPKSNK | 15 | 0.49 | 1.01 | 47694.91 | 2.21 |
| 01 | ACH | | | | | | | AYH | | | | | |

### Example 7

MHC Class II Vaccine Peptide 7

RFFPPKSNKA [symbol] HYHSYNGWNR (SEQ ID NO: 1)

| Transcript name | DNAJC10-001 |
|---|---|
| Length | 21 |
| MHC Class I immunogenicity score | 0.003 |
| MHC Class II immunogenicity score | 0.607 |
| RNA TPM | 0 |
| Max coding sequence coverage | 155 |
| Mutant amino acids | 1 |
| Mutation distance from edge | 10 |

Predicted mutant epitopes

| | | | | | | | WT | WT |
|---|---|---|---|---|---|---|---|---|
| | | SEQ | Binding | Binding | | SEQ | binding | binding |
| MHC | | ID | affinity | probability | | ID | affinity | probability |
| allele | Sequence | NO: | (nM) | (%) | WT sequence | NO: | (nM) | (nM) |
| DQB105:01 | RFFPPKSNKACHYHSYNGW | 1 | 10548.43 | 7.22 | RFFPPKSNKAYHYHSYNGW | 20 | 9545.80 | 7.47 |
| | NR | | | | NR | | | |
| DQB103:02 | RFFPPKSNKACHYHSYNGW | 1 | 204.27 | 25.36 | RFFPPKSNKAYHYHSYNGW | 20 | 198.50 | 25.56 |
| | NR | | | | NR | | | |
| DRB101:03 | RFFPPKSNKACHYHSYNGW | 1 | 25858.15 | 5.27 | RFFPPKSNKAYHYHSYNGW | 20 | 19298.17 | 5.85 |
| | NR | | | | NR | | | |
| DPA101:03 | RFFPPKSNKACHYHSYNGW | 1 | 13682.92 | 6.6 | RFFPPKSNKAYHYHSYNGW | 20 | 15056.78 | 6.38 |
| | NR | | | | NR | | | |
| DQA103:01 | RFFPPKSNKACHYHSYNGW | 1 | 35896.85 | 4.69 | RFFPPKSNKAYHYHSYNGW | 20 | 30768.04 | 4.96 |
| | NR | | | | NR | | | |
| DQA101:01 | RFFPPKSNKACHYHSYNGW | 1 | 3934.80 | 10.11 | RFFPPKSNKAYHYHSYNGW | 20 | 8640.81 | 7.74 |
| | NR | | | | NR | | | |
| DRB104:01 | RFFPPKSNKACHYHSYNGW | 1 | 4130.04 | 9.95 | RFFPPKSNKAYHYHSYNGW | 20 | 2760.52 | 11.38 |
| | NR | | | | NR | | | |
| DRB401:03 | RFFPPKSNKACHYHSYNGW | 1 | 1917.02 | 12.83 | RFFPPKSNKAYHYHSYNGW | 20 | 3590.63 | 10.43 |
| | NR | | | | NR | | | |
| DPB102:01 | RFFPPKSNKACHYHSYNGW | 1 | 1688.37 | 13.37 | RFFPPKSNKAYHYHSYNGW | 20 | 1004.77 | 15.78 |
| | NR | | | | NR | | | |
| DPB103:01 | RFFPPKSNKACHYHSYNGW | 1 | 14065.06 | 6.53 | RFFPPKSNKAYHYHSYNGW | 20 | 13310.95 | 6.66 |
| | NR | | | | NR | | | |

In Examples 6 and 7 shown above, short sequence “KACHYHSYNGW” of Example 2 is used to create a sequence for the long MHC Class I vaccine peptide of Example 6 and the long MHC Class II vaccine peptide of Example 7, both including the same short subsequence as Example 2 (e.g., the boxed letter “C”) at the center of the sequence. Predicted mutant epitopes may be generated or determined for both the MHC Class I vaccine peptide and the MHC Class II vaccine peptide, along with corresponding immunogenicity scores. The MHC Class I immunogenicity score may indicate a probability that at least one epitope in a longer peptide sequence is immunogenic on at least one of the subject's MHC Class I alleles. The MHC Class II immunogenicity score may indicate a probability that at least one epitope in a longer peptide sequence is immunogenic on at least one of the subject's MHC Class II alleles.

### Example 8

| Variant | chr17 g.17380542T > C |
|---|---|
| IGV locus | chr17: 17380542 |
| Gene name | MED9 |
| Gene ID | ENSG00000141026 |
| RNA reads supporting variant allele | 20 |
| RNA reads supporting reference allele | 0 |
| RNA reads supporting other alleles | 0 |
| RNA TPM | 0 |
| Cluster ID | 15 |
| Cluster Assignment Probability | 0.113 |
| Cellular Prevalence | 0.99 |

Predicted Effect

| | |
|---|---|
| Effect type | Substitution |
| Transcript name | MED9-001 |
| Transcript ID | ENST00000268711 |
| Effect description | p.Y63H |

MHC Class I Vaccine Peptide

(SEQ ID NO: 49)
RAREEEN [symbol] SFLPLVHNII

| | |
|---|---|
| Length | 18 |
| MHC Class I immunogenicity score | 0.002 |
| MHC Class I immunogenicity-binding score | 0.001 |
| MHC Class I unscaled immunogenicity score | 0.153 |
| MHC Class I unscaled immunogenicity-binding score | 0.112 |
| MHC Class I binding score | 1.0 |
| RNA TPM | 0 |
| Max coding sequence coverage | 19 |
| Mutant amino acids | 1 |
| Mutation distance from edge | 7 |

Predicted mutant epitopes

| | | | | | | | | | | WT | | | |
|---|---|---|---|---|---|---|---|---|---|---|---|---|---|
| | | | | Immuno- | Presen- | | | | | immuno- | | | WT |
| | | | Dis- | genicity | tation | | Binding | | | genicity | | WT | binding |
| | | SEQ | tance | prob- | prob- | Binding | prob- | | SEQ | prob- | WT | binding | prob- |
| MHC | | ID | to | ability | ability | affinity | ability | WT | ID | ability | presen- | affinity | ability |
| allele | Sequence | NO: | self | (%) | (%) | (nM) | (%) | sequence | NO: | (%) | tation | (nM) | (nM) |
| A*02: | REEENHSFL | 50 | 5 | 0.66 | 44.12 | 5351.98 | 13.66 | REEENYSFL | 59 | 0.56 | 38.94 | 4222.01 | 16.34 |
| 01 | | | | | | | | | | | | | |
| A*02: | EEENHSFLP | 51 | 5 | 0.72 | 46.19 | 10075.12 | 8.27 | EEENYSFLP | 60 | 0.5 | 37.14 | 12437.68 | 6.95 |
| 01 | L | | | | | | | L | | | | | |
| A*02: | HSFLPLVHN | 52 | 5 | 1.04 | 52.37 | 6841.69 | 11.28 | YSFLPLVHN | 61 | 4.89 | 70.46 | 2579.40 | 23.23 |
| 01 | I | | | | | | | I | | | | | |
| A*02: | ENHSFLPLV | 53 | 5 | 1.25 | 54.78 | 6143.91 | 12.27 | ENYSFLPLV | 62 | 3.33 | 66.19 | 2303.71 | 25.07 |
| 01 | | | | | | | | | | | | | |
| C*03: | HSFLPLVH | 54 | 5 | 3.07 | 65.29 | 2276.44 | 25.27 | YSFLPLVH | 63 | 24.39 | 89.72 | 1214.87 | 37.15 |
| 04 | | | | | | | | | | | | | |
| C*03: | RAREEENHS | 55 | 5 | 2.4 | 62.53 | 998.36 | 41.3 | RAREEENYS | 64 | 2.89 | 64.6 | 1121.96 | 38.81 |
| 04 | FL | | | | | | | FL | | | | | |
| C*03: | ENHSFLPL | 56 | 5 | 0.51 | 33.44 | 2722.72 | 22.38 | ENYSFLPL | 65 | 0.55 | 37.75 | 3824.50 | 17.57 |
| 04 | | | | | | | | | | | | | |
| B*58: | RAREEENHS | 57 | 5 | 27.26 | 91.19 | 309.65 | 66.58 | RAREEENYS | 66 | 39.47 | 96.21 | 115.97 | 82.67 |
| 01 | F | | | | | | | F | | | | | |
| B*58: | EEENHSFLP | 51 | 5 | 0.49 | 19.03 | 35515.61 | 2.86 | EEENYSFLP | 60 | 0.49 | 17.42 | 39616.24 | 2.60 |
| 01 | L | | | | | | | L | | | | | |
| B*58: | HSFLPLVHN | 52 | 5 | 6.96 | 74.43 | 3493.24 | 18.77 | YSFLPLVHN | 61 | 2.65 | 63.66 | 3673.45 | 18.10 |
| 01 | I | | | | | | | I | | | | | |
| C*05: | RAREEENHS | 55 | 5 | 5.85 | 72.47 | 635.96 | 51.24 | RAREEENYS | 64 | 10.79 | 79.55 | 1024.76 | 40.74 |
| 01 | FL | | | | | | | FL | | | | | |
| C*05: | HSFLPLVHN | 58 | 5 | 4.28 | 68.98 | 1365.94 | 34.75 | YSFLPLVHN | 67 | 3.8 | 67.65 | 1946.19 | 27.99 |
| 01 | II | | | | | | | II | | | | | |
| A*03: | HSFLPLVH | 54 | 5 | 1.88 | 59.77 | 23779.07 | 4.03 | YSFLPLVH | 63 | 0.73 | 46.37 | 29311.51 | 3.37 |
| 01 | | | | | | | | | | | | | |
| A*03: | RAREEENHS | 55 | 5 | 0.49 | 14.81 | 37482.34 | 2.73 | RAREEENYS | 64 | 0.49 | 12.31 | 37875.33 | 2.70 |
| 01 | FL | | | | | | | FL | | | | | |
| B*40: | REEENHSFL | 50 | 5 | 51.54 | 99.99 | 4.38 | 98.87 | REEENYSFL | 59 | 51.49 | 99.98 | 3.61 | 99.05 |
| 01 | | | | | | | | | | | | | |
| B*40: | HSFLPLVHN | 58 | 5 | 0.49 | 9.28 | 32162.34 | 3.11 | YSFLPLVHN | 67 | 0.49 | 6.16 | 36230.94 | 2.81 |
| 01 | II | | | | | | | II | | | | | |

### Example 9

MHC Class II Vaccine Peptide

QSPARAREEEN [symbol] SFLPLVHNII (SEQ ID NO: 68)

| Transcript name | MED9-001 |
|---|---|
| Length | 22 |
| MHC Class I immunogenicity score | 0.002 |
| MHC Class II immunogenicity score | 0.8 |
| RNATPM | 0 |
| Max coding sequence coverage | 19 |
| Mutant amino acids | 1 |
| Mutation distance from edge | 10 |

Predicted Mutant Epitopes

| | | | | Binding | | | WT | WT |
|---|---|---|---|---|---|---|---|---|
| | | SEQ | Binding | prob- | | SEQ | binding | binding |
| MHC | | ID | affinity | ability | | ID | affinity | probability |
| allele | Sequence | NO: | (nM) | (%) | WT sequence | NO: | (nM) | (nM) |
| DQB105:01 | QSPARAREEENHSFLPLV | 68 | 2829.97 | 11.29 | QSPARAREEENYSFLPLV | 69 | 1756.28 | 13.20 |
| | HNII | | | | HNII | | | |
| DQB103:02 | QSPARAREEENHSFLPLV | 68 | 125.84 | 28.94 | QSPARAREEENYSFLPLV | 69 | 67.71 | 33.92 |
| | HNII | | | | HNII | | | |
| DRB101:03 | QSPARAREEENHSFLPLV | 68 | 4001.83 | 10.05 | QSPARAREEENYSFLPLVI | 69 | 942.78 | 16.10 |
| | HNII | | | | HNII | | | |
| DPA101:03 | QSPARAREEENHSFLPLV | 68 | 179.97 | 26.27 | QSPARAREEENYSFLPLV | 69 | 31.59 | 40.56 |
| | HNII | | | | HNII | | | |
| DQA103:01 | QSPARAREEENHSFLPLV | 68 | 2340.43 | 12.02 | QSPARAREEENYSFLPLV | 69 | 2308.74 | 12.07 |
| | HNII | | | | HNII | | | |
| DQA101:01 | QSPARAREEENHSFLPLV | 68 | 736.57 | 17.38 | QSPARAREEENYSFLPLV | 69 | 630.98 | 18.23 |
| | HNII | | | | HNII | | | |
| DRB104:01 | QSPARAREEENHSFLPLV | 68 | 5988.97 | 8.77 | QSPARAREEENYSFLPLV | 69 | 2156.34 | 12.35 |
| | HNII | | | | HNII | | | |
| DRB401:03 | QSPARAREEENHSFLPLV | 68 | 48.46 | 36.77 | QSPARAREEENYSFLPLV | 69 | 19.12 | 45.15 |
| | HNII | | | | HNII | | | |
| DPB102:01 | QSPARAREEENHSFLPLV | 68 | 32.95 | 40.18 | QSPARAREEENYSFLPLV | 69 | 16.51 | 46.52 |
| | HNII | | | | HNII | | | |
| DPB103:01 | QSPARAREEENHSFLPLV | 68 | 74.04 | 33.17 | QSPARAREEENYSFLPLV | 69 | 34.11 | 39.87 |
| | HNII | | | | HNII | | | |

In Examples 8 and 9 shown above, short sequence “REEENHSFL” of Example 3 is used to create a sequence for the long MHC Class I vaccine peptide of Example 8 and the long MHC Class II vaccine peptide of Example 9, both including the same short subsequence as Example 3 (e.g., boxed letter “H”) at the center of the sequence. Predicted mutant epitopes may be generated or determined for both the MHC Class I vaccine peptide and the MHC Class II vaccine peptide, along with corresponding immunogenicity scores. The MHC Class I immunogenicity score may indicate a probability that at least one epitope in a longer peptide sequence is immunogenic on at least one of the subject's MHC Class I alleles. The MHC Class II immunogenicity score may indicate a probability that at least one epitope in a longer peptide sequence is immunogenic on at least one of the subject's MHC Class II alleles.

1. A method for ranking tumor-specific neoantigens from a tumor of a subject for a subject-specific immunogenic composition, comprising:
a) identifying a plurality of somatic mutations present in the tumor;
b) for an individual somatic mutation in the plurality of somatic mutations:
i) determining a best short neoantigen from an initial plurality of short neoantigens based at least in part on an immunogenicity score of the best short neoantigen;
ii) determining a best long neoantigen from an initial plurality of long neoantigens based at least in part on an immunogenicity score of the best long neoantigen;
iii) adding the best short neoantigen to a list of short neoantigen candidates; and
iv) adding the best long neoantigen to a list of long neoantigen candidates;
c) performing step b for the plurality of somatic mutations, wherein the list of short neoantigen candidates when completed includes the respective best short neoantigens for the plurality of somatic mutations, and wherein the list of long neoantigen candidates when completed includes the respective best long neoantigens for the plurality of somatic mutations;
d) ranking the list of short neoantigen candidates by descending immunogenicity score; and
e) ranking the list of long neoantigen candidates by descending immunogenicity score.

2. The method of claim 1, further comprising:
identifying, for the individual somatic mutation, a longest neoantigen sequence that includes a mutated amino acid;
identifying the initial plurality of short neoantigens from the longest neoantigen sequence, wherein individual neoantigens in the initial plurality of short neoantigens include the mutated amino acid and have between a minimum and maximum number of amino acids; and
for an individual allele in a plurality of HLA class I alleles present in the subject, determining respective neoantigen-allele scores for the initial plurality of short neoantigens.

3. (canceled)

4. (canceled)

5. The method of claim 2, wherein the neoantigen-allele score for an individual neoantigen of the initial plurality of short neoantigens and the individual allele is based at least in part on a probability that the individual neoantigen is presented by the individual allele and a germline sibling of the individual neoantigen is not presented by the individual allele, and wherein the probability is determined at least in part based on data from an MHC Class I machine learning model trained to determine a probability that a given allele in the plurality of HLA class I alleles presents a certain antigen.

6. (canceled)

7. The method of claim 2, further comprising:
for any two neoantigens in the initial plurality of short neoantigens wherein one of the two neoantigens includes the other of the two neoantigens, removing, from the initial plurality of short neoantigens, the neoantigen of the two neoantigens that has a lower neoantigen-allele score for the individual allele.

8. The method of claim 7, further comprising:
identifying a short subsequence, the short subsequence being the shortest subsequence of the longest neoantigen sequence that includes all of the neoantigens in the initial plurality of short neoantigens, wherein no neoantigen in the initial plurality of short neoantigens is included in another neoantigen in the initial plurality of short neoantigens; and
determining a probability that the individual allele presents at least one neoantigen in the set of short neoantigens and does not present a germline sibling of the least one neoantigen.

9. (canceled)

10. The method of claim 2, further comprising:
determining the immunogenicity score of an individual neoantigen in the set of short neoantigens, the immunogenicity score based at least in part on a probability that at least one allele in a plurality of HLA class I alleles of the subject presents the individual neoantigen and does not present a germline sibling of the individual neoantigen.

11. The method of claim 2, further comprising:
determining a probability that at least one allele in a plurality of HLA class I alleles of the subject presents at least one neoantigen in the set of short neoantigens and does not present a germline sibling of the at least one neoantigen.

12. The method of claim 8, further comprising:
identifying an expanded sequence, the expanded sequence being a subsequence of the longest neoantigen that includes the short subsequence and a first maximum number of amino acids on each side of the mutated amino acid; and
identifying a set of long neoantigens from the expanded sequence, the set of long neoantigens having lengths ranging between the length of the short subsequence and a second maximum number of amino acids; and
removing any neoantigens from the set of long neoantigens that do not satisfy a manufacturability condition,
wherein the first maximum number is 29, and wherein the second maximum number is 30.

13-15. (canceled)

16. The method of claim 12, further comprising:
determining the immunogenicity score of an individual neoantigen in the set of long neoantigens, wherein the immunogenicity score is based at least in part on a probability that at least one allele in a plurality of HLA class II alleles of the subject presents the individual neoantigen and does not present a germline sibling of the individual neoantigen, wherein the probability is determined based at least in part on data from an MHC Class II machine learning model trained to determine a probability that a given allele in the plurality of HLA class II alleles presents a certain antigen, and wherein the immunogenicity scores is determined at least in part based on data from a machine learning model.

17. (canceled)

18. (canceled)

19. The method of claim 1, further comprising:
providing the list of long neoantigen candidates for manufacturability analysis; and
receiving a subset of long neoantigen candidates, the subset of long neoantigen candidates selected from the list of long neoantigen candidates based at least in part on manufacturability.

20. (canceled)

21. The method of claim 1, further comprising:
selecting a subset of long neoantigen candidates from the list of long neoantigen candidates based at least in part on manufacturability.

22. The method of claim 21, further comprising:
removing, from the list of short neoantigen candidates, any neoantigens that are included in any of the subset of long neoantigen candidates.

23. The method of claim 1, further comprising:
trimming the list of short neoantigen candidates to a predetermined number of top short neoantigen candidates based on immunogenicity score.

24. The method of claim 1, further comprising:
providing the list of short neoantigen candidates for manufacturability analysis; and
receiving a subset of short neoantigen candidates, the subset of short neoantigen candidates selected from the list of short neoantigen candidates based at least in part on manufacturability.

25. (canceled)

26. (canceled)

27. The method of claim 1, further comprising:
forming a subject-specific immunogenic composition comprising one or more neoantigens from the list of short neoantigen candidates; and
forming a subject-specific immunogenic composition comprising one or more neoantigens from the list of long neoantigen candidates.

28. (canceled)

29. (canceled)

30. The method of claim 1, wherein the initial plurality of short neoantigens comprises short polypeptides that include at least one MHC Class I epitope associated with the subject;
wherein the initial plurality of long neoantigens comprises long polypeptides that include at least one MHC Class I epitope and at least one MHC Class II epitope associated with the subject; and
wherein the initial plurality of short neoantigens and the initial plurality of long neoantigens are derived from the tumor and include the individual somatic mutation.

31-33. (canceled)

34. The method of claim 1, wherein the best short neoantigen for the individual somatic mutation is the short neoantigen with the highest immunogenicity score of all the initial plurality of short neoantigens with respect to the individual somatic mutation, and
wherein the best long neoantigen for the individual somatic mutation is the long neoantigen with the highest immunogenicity score of all the initial plurality of long neoantigens with respect to the individual somatic mutation.

35. (canceled)

36. A method for ranking tumor-specific neoantigens from a tumor of a subject for a subject-specific immunogenic composition, comprising:
a) identifying a plurality of somatic mutations present in the tumor;
b) for an individual somatic mutation in the plurality of somatic mutations:
i) determining a best short neoantigen from an initial plurality of short neoantigens based at least in part on a quality score of the best short neoantigen, wherein the quality score is based at least in part on at least one selected from the group of predicted presentation probability, predicted binding affinity, and predicted immunogenic response;
ii) determining a best long neoantigen from an initial plurality of long neoantigens based at least in part on a quality score of the best long neoantigen, wherein the quality score is based at least in part on at least one selected from the group of predicted presentation probability, predicted binding affinity, and predicted immunogenic response;
iii) adding the best short neoantigen to a list of short neoantigen candidates; and
iv) adding the best long neoantigen to a list of long neoantigen candidates;
c) performing step b for the plurality of somatic mutations, wherein the list of short neoantigen candidates when completed includes the respective best short neoantigens for the plurality of somatic mutations, and wherein the list of long neoantigen candidates when completed includes the respective best long neoantigens for the plurality of somatic mutations;
d) ranking the list of short neoantigen candidates based at least in part on a ranking algorithm that includes quality score; and
e) ranking the list of long neoantigen candidates based at least in part on the ranking algorithm or a second ranking algorithm that includes quality score.

37. (canceled)

38. (canceled)

39. The method of claim 36, wherein the predicted binding affinity is determined based at least in part on data from an MHC Class II learning model trained to determine the binding affinity between a Class II HLA allele and a given peptide.

40. (canceled)

41. (canceled)

42. The method of claim 36, wherein the predicted presentation probability, predicted binding affinity, and predicted presentation probability are determined by one or more machine learning models.


SEQUENCE LISTING (69 sequences)

SEQ ID NO: 1 - protein, 21 aa, Artificial Sequence
  Description of Artificial Sequence Synthetic peptide
RFFPPKSNKACHYHSYNGWNR

SEQ ID NO: 2 - protein, 11 aa, Artificial Sequence
  Description of Artificial Sequence Synthetic peptide
FPPKSNKACHY

SEQ ID NO: 3 - protein, 8 aa, Artificial Sequence
  Description of Artificial Sequence Synthetic peptide
KACHYHSY

SEQ ID NO: 4 - protein, 8 aa, Artificial Sequence
  Description of Artificial Sequence Synthetic peptide
CHYHSYNG

SEQ ID NO: 5 - protein, 8 aa, Artificial Sequence
  Description of Artificial Sequence Synthetic peptide
SNKACHYH

SEQ ID NO: 6 - protein, 11 aa, Artificial Sequence
  Description of Artificial Sequence Synthetic peptide
FFPPKSNKACH

SEQ ID NO: 7 - protein, 11 aa, Artificial Sequence
  Description of Artificial Sequence Synthetic peptide
KACHYHSYNGW

SEQ ID NO: 8 - protein, 11 aa, Artificial Sequence
  Description of Artificial Sequence Synthetic peptide
RFFPPKSNKAC

SEQ ID NO: 9 - protein, 11 aa, Artificial Sequence
  Description of Artificial Sequence Synthetic peptide
KSNKACHYHSY

SEQ ID NO: 10 - protein, 11 aa, Artificial Sequence
  Description of Artificial Sequence Synthetic peptide
CHYHSYNGWNR

SEQ ID NO: 11 - protein, 11 aa, Homo sapiens
FPPKSNKAYHY

SEQ ID NO: 12 - protein, 8 aa, Homo sapiens
KAYHYHSY

SEQ ID NO: 13 - protein, 8 aa, Homo sapiens
YHYHSYNG

SEQ ID NO: 14 - protein, 8 aa, Homo sapiens
SNKAYHYH

SEQ ID NO: 15 - protein, 11 aa, Homo sapiens
FFPPKSNKAYH

SEQ ID NO: 16 - protein, 11 aa, Homo sapiens
KAYHYHSYNGW

SEQ ID NO: 17 - protein, 11 aa, Homo sapiens
RFFPPKSNKAY

SEQ ID NO: 18 - protein, 11 aa, Homo sapiens
KSNKAYHYHSY

SEQ ID NO: 19 - protein, 11 aa, Homo sapiens
YHYHSYNGWNR

SEQ ID NO: 20 - protein, 21 aa, Homo sapiens
RFFPPKSNKAYHYHSYNGWNR

SEQ ID NO: 21 - protein, 19 aa, Artificial Sequence
  Description of Artificial Sequence Synthetic peptide
HEHWRFVLQHLVFLAAFVV

SEQ ID NO: 22 - protein, 9 aa, Artificial Sequence
  Description of Artificial Sequence Synthetic peptide
WRFVLQHLV

SEQ ID NO: 23 - protein, 9 aa, Artificial Sequence
  Description of Artificial Sequence Synthetic peptide
VLQHLVFLA

SEQ ID NO: 24 - protein, 10 aa, Artificial Sequence
  Description of Artificial Sequence Synthetic peptide
EHWRFVLQHL

SEQ ID NO: 25 - protein, 9 aa, Artificial Sequence
  Description of Artificial Sequence Synthetic peptide
HLVFLAAFV

SEQ ID NO: 26 - protein, 9 aa, Artificial Sequence
  Description of Artificial Sequence Synthetic peptide
FVLQHLVFL

SEQ ID NO: 27 - protein, 8 aa, Artificial Sequence
  Description of Artificial Sequence Synthetic peptide
WRFVLQHL

SEQ ID NO: 28 - protein, 9 aa, Artificial Sequence
  Description of Artificial Sequence Synthetic peptide
EHWRFVLQH

SEQ ID NO: 29 - protein, 10 aa, Artificial Sequence
  Description of Artificial Sequence Synthetic peptide
HLVFLAAFVV

SEQ ID NO: 30 - protein, 9 aa, Artificial Sequence
  Description of Artificial Sequence Synthetic peptide
LQHLVFLAA

SEQ ID NO: 31 - protein, 9 aa, Artificial Sequence
  Description of Artificial Sequence Synthetic peptide
RFVLQHLVF

SEQ ID NO: 32 - protein, 9 aa, Artificial Sequence
  Description of Artificial Sequence Synthetic peptide
HWRFVLQHL

SEQ ID NO: 33 - protein, 8 aa, Artificial Sequence
  Description of Artificial Sequence Synthetic peptide
VLQHLVFL

SEQ ID NO: 34 - protein, 11 aa, Artificial Sequence
  Description of Artificial Sequence Synthetic peptide
HEHWRFVLQHL

SEQ ID NO: 35 - protein, 9 aa, Homo sapiens
WRFVLQRLV

SEQ ID NO: 36 - protein, 9 aa, Homo sapiens
VLQRLVFLA

SEQ ID NO: 37 - protein, 10 aa, Homo sapiens
EHWRFVLQRL

SEQ ID NO: 38 - protein, 9 aa, Homo sapiens
RLVFLAAFV

SEQ ID NO: 39 - protein, 9 aa, Homo sapiens
FVLQRLVFL

SEQ ID NO: 40 - protein, 8 aa, Homo sapiens
WRFVLQRL

SEQ ID NO: 41 - protein, 9 aa, Homo sapiens
EHWRFVLQR

SEQ ID NO: 42 - protein, 10 aa, Homo sapiens
RLVFLAAFVV

SEQ ID NO: 43 - protein, 9 aa, Homo sapiens
LQRLVFLAA

SEQ ID NO: 44 - protein, 9 aa, Homo sapiens
RFVLQRLVF

SEQ ID NO: 45 - protein, 9 aa, Homo sapiens
HWRFVLQRL

SEQ ID NO: 46 - protein, 8 aa, Homo sapiens
VLQRLVFL

SEQ ID NO: 47 - protein, 11 aa, Homo sapiens
HEHWRFVLQRL

SEQ ID NO: 48 - protein, 19 aa, Homo sapiens
HEHWRFVLQRLVFLAAFVV

SEQ ID NO: 49 - protein, 18 aa, Artificial Sequence
  Description of Artificial Sequence Synthetic peptide
RAREEENHSFLPLVHNII

SEQ ID NO: 50 - protein, 9 aa, Artificial Sequence
  Description of Artificial Sequence Synthetic peptide
REEENHSFL

SEQ ID NO: 51 - protein, 10 aa, Artificial Sequence
  Description of Artificial Sequence Synthetic peptide
EEENHSFLPL

SEQ ID NO: 52 - protein, 10 aa, Artificial Sequence
  Description of Artificial Sequence Synthetic peptide
HSFLPLVHNI

SEQ ID NO: 53 - protein, 9 aa, Artificial Sequence
  Description of Artificial Sequence Synthetic peptide
ENHSFLPLV

SEQ ID NO: 54 - protein, 8 aa, Artificial Sequence
  Description of Artificial Sequence Synthetic peptide
HSFLPLVH

SEQ ID NO: 55 - protein, 11 aa, Artificial Sequence
  Description of Artificial Sequence Synthetic peptide
RAREEENHSFL

SEQ ID NO: 56 - protein, 8 aa, Artificial Sequence
  Description of Artificial Sequence Synthetic peptide
ENHSFLPL

SEQ ID NO: 57 - protein, 10 aa, Artificial Sequence
  Description of Artificial Sequence Synthetic peptide
RAREEENHSF

SEQ ID NO: 58 - protein, 11 aa, Artificial Sequence
  Description of Artificial Sequence Synthetic peptide
HSFLPLVHNII

SEQ ID NO: 59 - protein, 9 aa, Homo sapiens
REEENYSFL

SEQ ID NO: 60 - protein, 10 aa, Homo sapiens
EEENYSFLPL

SEQ ID NO: 61 - protein, 10 aa, Homo sapiens
YSFLPLVHNI

SEQ ID NO: 62 - protein, 9 aa, Homo sapiens
ENYSFLPLV

SEQ ID NO: 63 - protein, 8 aa, Homo sapiens
YSFLPLVH

SEQ ID NO: 64 - protein, 11 aa, Homo sapiens
RAREEENYSFL

SEQ ID NO: 65 - protein, 8 aa, Homo sapiens
ENYSFLPL

SEQ ID NO: 66 - protein, 10 aa, Homo sapiens
RAREEENYSF

SEQ ID NO: 67 - protein, 11 aa, Homo sapiens
YSFLPLVHNII

SEQ ID NO: 68 - protein, 22 aa, Artificial Sequence
  Description of Artificial Sequence Synthetic peptide
QSPARAREEENHSFLPLVHNII

SEQ ID NO: 69 - protein, 22 aa, Homo sapiens
QSPARAREEENYSFLPLVHNII